(19) United States
(12) Patent Application Publication
(10) Pub. No.: US 2016/0038083 A1
(43) Pub. Date: Feb. 11, 2016

(54) GARMENT INCLUDING INTEGRATED SENSOR COMPONENTS AND FEEDBACK COMPONENTS

(71) Applicant: Orn, Inc., Cambridge, MA (US)

(72) Inventors: Ye Ding, Cambridge, MA (US); Arnar Freyr Larusson, Cambridge, MA (US)

(21) Appl. No.: 14/822,332

(22) Filed: Aug. 10, 2015

Related U.S. Application Data
(60) Provisional application No. 62/035,172, filed on Aug. 8, 2014.

(51) Int. Cl. A61B 5/00; A61B 5/11; A61B 5/024; A41D 13/12

(52) U.S. Cl. CPC A61B 5/6804; A41D 13/1281; A61B 5/1107; A61B 5/1121; A61B 5/486; A61B 5/7455; A61B 5/024; A61B 5/6844; A61B 2562/164; A61B 2560/0242

(57) ABSTRACT

A garment for measuring one or more parameters of a wearer includes a base material configured to be worn by a wearer and a sensing component. The sensing component has a first elastic stretchability along a first axis and a second elastic stretchability along a second axis that is greater than the first elastic stretchability. The sensing component is integrated into a first location of the base material corresponding to a predetermined region of the wearer. The sensing component includes an electrically conductive material having an electrical resistance that changes with a change in a length of the sensing component. The sensing component includes at least one wire to electrically couple the electrically conductive material to a controller including a processor and a memory. The memory stores processor-executable instructions to cause the controller to determine a electrical resistance value across the sensing component via the at least one wire.

### CROSS REFERENCE TO RELATED APPLICATIONS

This application claims the benefit of and priority to U.S. Provisional Patent Application No. 62/035,172, filed Aug. 8, 2014 and entitled “Systems and Methods for Garment Integrated Device to Monitor Wearer Information and Provide Real-Time Feedback”, the entire disclosure of which is incorporated herein by reference.

### FIELD OF THE DISCLOSURE

The present application relates generally to garments and more particularly, to garments including integrated sensing components and feedback components.

### BACKGROUND

There is an increased interest, among individuals, healthcare providers, fitness facilities, and others, in tracking individual biometric and physiometric data. Some existing mobile handsets and respective mobile applications provide some platforms to record and track user related data. However, the platforms provided by mobile handsets have thus far been inadequate.

### SUMMARY

The present disclosure relates to a system that utilizes a garment including integrated sensing components and feedback components. The system can collect and process the wearer's location information and physiometric data from the sensors. The system can give real time feedback based on the collected data to the wearer through either haptic feedback, audio feedback or visual feedback, among others. The onboard processing and real time feedback can provide strategies based on the data collected and a training plan. The strategies can instruct the wearer through real time feedback to improve their performance by built-in customized training algorithms based on the wearer's historical data. In various embodiments, systems described herein include a stretchable garment that includes resistance sensors, accelerometers and/or gyroscopes, and an integrated controller and is configured to determine a breathing pattern, movement and/or posture or orientation of the wearer.

In one aspect, a garment for measuring one or more parameters of a wearer includes a base material configured to be worn by a wearer and a sensing component. The sensing component has a first elastic stretchability along a first axis and a second elastic stretchability along a second axis that is greater than the first elastic stretchability. The sensing component is integrated into a first location of the base material corresponding to a predetermined region of the wearer. The sensing component includes an electrically conductive material having an electrical resistance that changes with a change in a length of the sensing component. The sensing component includes at least one wire to electrically couple the electrically conductive material to a controller including a processor and a memory. The memory stores processor-executable instructions to cause the controller to determine an electrical resistance value across the sensing component via the at least one wire.

In some embodiments, the base material includes a torso portion configured to surround a torso of a wearer such that the first location of the base material is located within the torso portion of the garment. In various embodiments, the sensing component extends along a circumference of the torso portion and has a height below a predetermined threshold. In various embodiments, the base material is shaped and sized to form a shirt such that the first location of the base material into which the sensing component is integrated is positioned at a first distance from a neckline of the base material which is based on a size of the base material. In some embodiments, the garment includes an electrical port coupled to the at least one wire. The electrical port is positioned on a back portion of the garment that is configured to cover a back of the wearer. The garment includes an attachment mechanism to secure a device including the controller and a connector to the garment and establish a connection between the electrical port and the connector.

In various embodiments, the device includes a body orientation detection sensor and the attachment mechanism is positioned on the back portion of the garment that is aligned with a spinal column of the wearer when the garment is worn by the wearer. In some embodiments, the base material is shaped to be worn as a shirt and the sensing component includes a first sensing component integrated into the first location of the shirt that is a first distance from a neckline of the shirt. The first location corresponds to a pectoral region of the wearer when the wearer wears the shirt. The shirt also includes a second sensing component integrated into a second location of the shirt that is a second distance from the neckline of the shirt. The second location of the garment corresponding to an abdominal region of the wearer when the wearer wears the shirt.

In various embodiments, the first sensing component can be used to measure a contraction and expansion of a rib cage and chest cavity of a wearer. Furthermore, the second sensing component can be used to measure a contraction and expansion of an abdominal cavity of a wearer. In various embodiments, the sensing component includes a plurality of electrically conductive particles positioned between a first film and a second film. The second film can have a water solubility below a predetermined threshold. In various embodiments, at least one of the first film or the second film is permanently secured to the garment. In some embodiment, the sensing component includes a strip positioned in between a first film and a second film. At least one of the first film or the second film is permanently secured to the garment.

In some embodiments, the body orientation detection sensor includes an accelerometer, magnetometer or a gyroscope. The controller is configured to sample values from the accelerometer, magnetometer or the gyroscope at a predetermined frequency. The posture detection sensor is configured to communicate with the controller to determine a posture of the wearer. In various embodiments, the garment includes one or more haptic vibrators. The haptic vibrators are configured to receive a signal from the controller responsive to the controller detecting a trigger event based on the resistance value of the sensing component. In various embodiments, the one or more haptic vibrators are positioned at a second location of the garment corresponding to a location of a bone of the wearer when the wearer wears the garment. In various embodiments, the one or more haptic vibrators are positioned at a second location of the garment corresponding to a location of a collarbone of the wearer when the wearer wears the garment.

In some implementations, the sensing component is positioned at a location of the garment to determine one of i) an expansion or contraction of a muscle or ii) a change in an orientation of a joint.

In another aspect, a shirt for measuring one or more parameters of a wearer includes a base material configured to be worn by a wearer. A sensing component having a first elastic stretchability along a first axis and a second elastic stretchability along a second axis that is greater than the first elasticity is integrated into a first location of the base material corresponding to a predetermined region of a wearer. The sensing component includes an electrically conductive material having an electrical resistance that changes with a change in a length of the sensing component. The sensing component includes at least one wire to electrically couple the electrically conductive material to a controller including a processor and a memory. The memory storing processor-executable instructions to cause the controller to determine a electrical resistance value across the sensing component via the at least one wire.

In some embodiments, the sensing component extends along a circumference of the torso portion and has a height below a predetermined threshold. In various embodiments, an electrical port is coupled to the at least one wire and is positioned on a back portion of the garment that is configured to cover a back of the wearer. An attachment mechanism is provided to secure a device including the controller and a connector to the garment and establish a connection between the electrical port and the connector. In some embodiments, the device includes a posture detection sensor and the attachment mechanism is positioned on the back portion of the garment that is aligned with a spinal column of the wearer when the garment is worn by the wearer. In various embodiments, the sensing component includes a first sensing component integrated into the first location of the shirt that is a first distance from a neckline of the shirt. The first location corresponds to a pectoral region of the wearer when the wearer wears the shirt. Furthermore, the shirt also includes a second sensing component integrated into a second location of the shirt that is a second distance from the neckline of the shirt. The second location of the garment corresponds to an abdominal region of the wearer when the wearer wears the shirt.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

The foregoing and other features of the present disclosure will become more fully apparent from the following description and appended claims, taken in conjunction with the accompanying drawings. Understanding that these drawings depict only several embodiments in accordance with the disclosure and are therefore, not to be considered limiting of its scope, the disclosure will be described with additional specificity and detail through use of the accompanying drawings.

FIG. 1 is a diagram depicting a smart garment on which a device to monitor wearer information and provide real-time feedback is integrated.

FIG. 2 is a block diagram depicting an example embodiment of a monitoring device that can be integrated in a smart garment and capable of communicating with a computer device.

FIG. 3 is a diagram depicting a cross section of a printed circuit board.

FIG. 4A is a block diagram depicting an embodiment of a network environment comprising local devices in communication with remote devices.

FIGS. 4B-4D are block diagrams depicting embodiments of computers useful in connection with the methods and systems described herein.

FIG. 5A is a front view of a garment which includes a plurality of breathing sensors.

FIG. 5B is a side cross-section view of a sensing component integrated into the garment of FIG. 5A.

FIG. 6 is a back view of the garment of FIG. 5A.

FIG. 7 is a schematic side cross-section view of a housing configured to house a controller which can be included in the garment of FIGS. 5A and 6.

FIG. 8A-B are perspective views of various electrical couplers according to the embodiment which can be used to electrically couple the controller of FIG. 5A and FIGS. 6-7 to one or more sensing components included in the garment.

FIG. 9 is a schematic block diagram depicting embodiments of a control module which can include the controller of FIGS. 5A and 6-7.

FIG. 10A is a schematic block diagram depicting an embodiment of a network environment comprising local devices connected to remote devices.

FIG. 10B shows a block diagram of the garment monitoring system (GMS) 120

FIG. 11 is a schematic diagram showing various changes in resistance with changes in length of a breathing sensor.

FIG. 12 shows a resistance diagram for determining an overall resistance of a first sensing component and a second sensing component by measuring resistances between a plurality of locations on the breathing sensors and adding the individual resistances to determine an overall resistance of the breathing sensors.

FIG. 13 is a plot of resistance vs percent stretch of a breathing sensor including calibration resistance values determined once a wearer wears the garment, and actual or factory values observed on the breathing sensor when the garment was first manufactured.

FIG. 14 shows an examples first sensor signal obtained from a first sensing component, a second sensor signal obtained from a second sensing component and an augmented sensor signal obtained therefrom.

FIG. 15 is a schematic block diagram of a first sensing component signal and a second sensing component signal collection and analysis topology which can be used by any of the controllers described herein.

FIG. 16 is a plot of a first sensing component signal and a second sensing component signal illustrating a method of determining various breathing parameters of a wearer wearing any embodiment of the garment described herein on a torso thereof.

FIG. 17 is a resistance signal plot of a first sensing component positioned around a chest of a wearer and a second sensing component positioned around an abdomen of the wearer which are included in a garment worn on a torso of the wearer, and are used to qualitatively determine a breathing pattern of the wearer.

FIG. 18 is a schematic flow diagram of an embodiment of a method for determining a breathing pattern of a wearer using a garment which includes a first sensing component and a second sensing component.

Reference is made to the accompanying drawings throughout the following detailed description. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION OF VARIOUS EMBODIMENTS

The present disclosure relates to a system of sensors, actuators, microprocessors and batteries that are integrated on a garment. The system can collect and process the wearer's location information and physiometric data from the sensors. The system can give real time feedback based on the collected data to the wearer through either haptic feedback, audio feedback or visual feedback. onboard processing and real time feedback can provide strategies based on the data collected and a training plan. The strategies can instruct the wearer through real time feedback to improve their performance by built-in customized training algorithms based on the wearer's historical data. In various embodiments, systems described herein include a stretchable garments that includes resistance sensors, accelerometers and/or gyroscopes, and an integrated controller and is configured to determine a breathing pattern, movement and/or posture or orientation of the wearer.

For purposes of reading the description of the various embodiments below, the following descriptions of the sections of the specification and their respective contents may be helpful:

Section A describes embodiments of systems and methods for a smart garment.

Section B describes a network environment and computing environment which may be useful for practicing embodiments described herein.

Section C describes embodiment of a garment that includes a pair of sensing components integrated into the garment and configured to electrically couple to a controller.

As used herein, the words “user” or “wearer” are used interchangeably to refer to an individual wearing the any of the garments described herein for determining one or more physiometric parameters thereof.

A. Systems and Methods of a Smart Garment

Various embodiments disclosed herein are directed to systems and methods of a smart garment on which a device to monitor wearer information and provide real-time feedback is integrated.

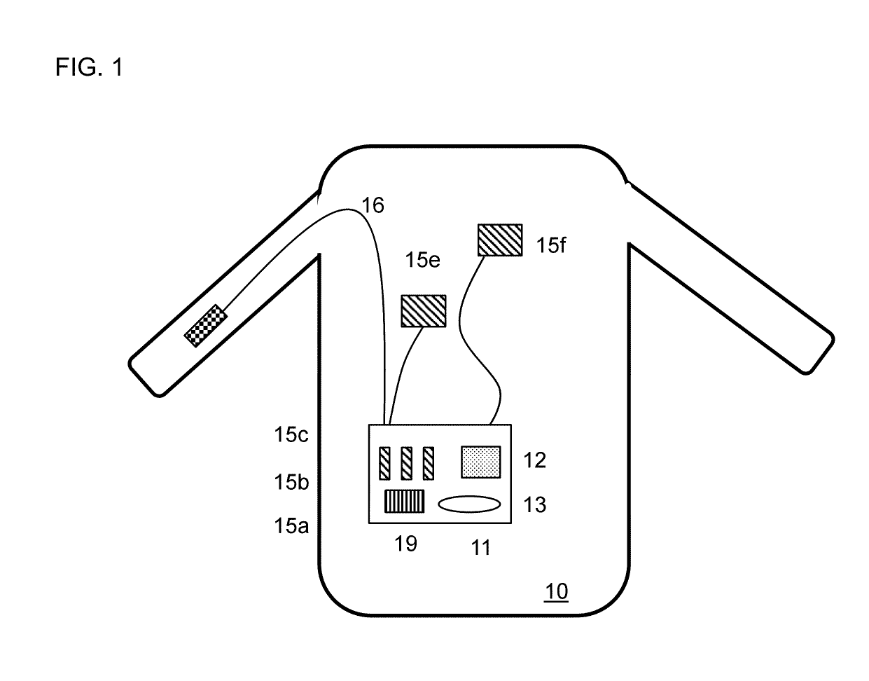

FIG. 1 is a diagram depicting a smart garment 10 on which a device to monitor wearer information and provide real-time feedback is integrated in accordance with various embodiments. As shown in FIG. 1, the smart garment 10 includes a microprocessor 12, a battery 13, a feedback device 14, a plurality of sensors 15a-15e, and a communication interface 19. The microprocessor 12, the battery 13, a subset of sensors 15a-15c, and the communication interface 19 are built in an integrated circuit (IC) 11. The smart garment also includes connecting wires 16.

While the smart garment 10 is depicted as a shirt in FIG. 1, a person of ordinary skill in the art should appreciate that examples of the smart garment 10 can include shorts, pants, swim wear, sportswear, or any other wearable garments.

In some embodiments, the plurality of sensors 15a-15e (also referred to hereinafter as sensors 15) include physiometric sensors, environmental sensors, and/or other types of sensors. The sensors 15 can be configured to measure motion signal (such as speed), heart rate, breath rate, dehydration rate, global position, sun exposure, light exposure, or other signals associated with the wearer (the individual wearing the garment). In some embodiments, the sensors 15 can perform measurements periodically when the IC 11 is activated. In some embodiments, the sensors 15 can be continuously performing measurements. In some embodiments, the sensors can perform measurements periodically, for example, every millisecond, second, minute, among other time units. The granularity of time between which a sensor may perform a measurement are measured may vary for different sensors.

In some embodiments, accelerometers can be used to measure motion signals. Accelerometers that are integrated in (or attached to) the textile of the smart garment 10 can be located on one or various parts of the wearer's body in order to detect states of motion. For instance, in physical training applications, one or more accelerometers can be placed on the back or chest of the wearer to measure the respective speed. Other accelerometers can be placed in/on the sleeves of the smart garment 10 to take measurements associated with the motion of the arms of the wearer. If the smart garment 10 is designed as a pair of pants, one or more accelerometers can be placed on the legs of the wearer to measure leg movement signals.

In some embodiments, a global positioning system (GPS) can be used to record location coordinates as the wearer moves from one location to another. The GPS can be implemented within the IC 11.

In some embodiments, electrocardiography (ECG) sensors can be used to measure the electrical activity of the wearer's heart. In other embodiments, heart pulse sensors can be employed to measure responses of the heart pulse wave of the wearer. The ECG sensors and/or the heart pulse sensors can be placed at different locations of the smart garment 10. For instance, one ECG or heart pulse sensor can be placed close to heart of the wearer.

In some embodiments, the sensors 15 include a salinity sensor configured to measure the salinity of the wearer's sweat. The salinity level of the wearer's sweat can be used to calculate or deduce a dehydration level of the wearer.

In some embodiments, the sensors 15 can include a conductivity sensor configured to measure the conductivity of the wearer's skin. Measured skin conductivity can provide an indication of the stress level of the wearer. Other physiometric sensors that can be integrated in (or attached to) the smart garment 10 include digital thermometers to measure skin temperature, blood oxygen sensors, and/or the like.

Environmental sensors that can be integrated in the smart garment 10 include a light sensor configured to measure ambient light (or sun light), humidity sensor configured to measure air humidity, temperature sensor configured to measure air temperature, atmospheric/environment pressure sensor to measure atmospheric (relevant to mountaineers) or water (relevant to divers) pressure, and/or the like.

The sensors 15 are configured to send signal measurements to the microprocessor 12. The microprocessor 12 can include internal memory (such as level 1 and/or level 2 cache) to store measurement values recorded by the sensors 15. In some embodiments, the microprocessor 12 can store (or have access to) other data of the wearer, such as physical training data, medical data, or the like. In some embodiments, the microprocessor 12 is configured to process the received measurements and form real-time decisions for presenting to the wearer. In some embodiments, the microprocessor 12 is configured to cause the transfer of the data collected by the sensors 15 and/or data deduced therefrom to a computer device such as a client device or server (e.g., the computer device 100 described herein). The transfer of the data can be performed periodically (such as every day) or in real time.

The feedback component 14 is configured to receive a signal (such as a signal indicative of a decision, instructions, biometric values, training performance metric values, or the like) from the microprocessor 12 and generate a feedback signal to the wearer. The feedback signal can be a haptic feedback signal (vibration motors, thermal stimulates, and/or the like), audio feedback signal, a visual feedback signal (led lights or signal displayed on a display), the like, or combinations thereof.

The communication interface 19 is configured to communicate with a computer device. The communication interface 19 allows the microprocessor 12 (or the IC 11) to send/receive commands or upload/download data to/from a client device (such as a smart phone, a PC, a tablet, or the like) or computer server. The communication interface 19 can be a Bluetooth interface, a wireless communication interface, a wired communication interface (such as USB interface), a near field communication (NFC) interface, the like, or a combination thereof.

The battery 13 is a power source for the IC 11 and other electronic components integrated off the IC 11 (such as the sensors 15a-5f and the feedback component 14). In some embodiments, the battery 13 can rechargeable. In other embodiments, the battery 13 can be replaceable.

The electronic components integrated off the IC 11 (such as the sensors 15e-15f and the feedback component 14) are coupled to the IC 11 through connecting wires (electric wires) 16. In some embodiments, the connecting wires 16 are integrated in the garment textile. In some embodiments, all the electronic components except the feedback components (such as the feedback component 14) are assembled on the IC 11. In other embodiments, the feedback component 14 can include a detachable component that can be coupled/decoupled to/from the IC 11 through connecting wires 16. For instance, a detachable component can include a smart watch, a wrist-wearable display (such as a display mounted on a wrist belt or watch belt), a wrist-wearable audio device, or the like.

In some embodiments, the IC 11 includes a printed circuit board (PCB). The PCB can include a thin insulating polymer film having conductive circuit patterns affixed thereto and supplied with a thin polymer coating to protect the conductor/electric circuits. The electric circuits can be formed by etching metal foil cladding (normally of copper) from polymer bases, plating metal or printing of conductive inks among other processes.

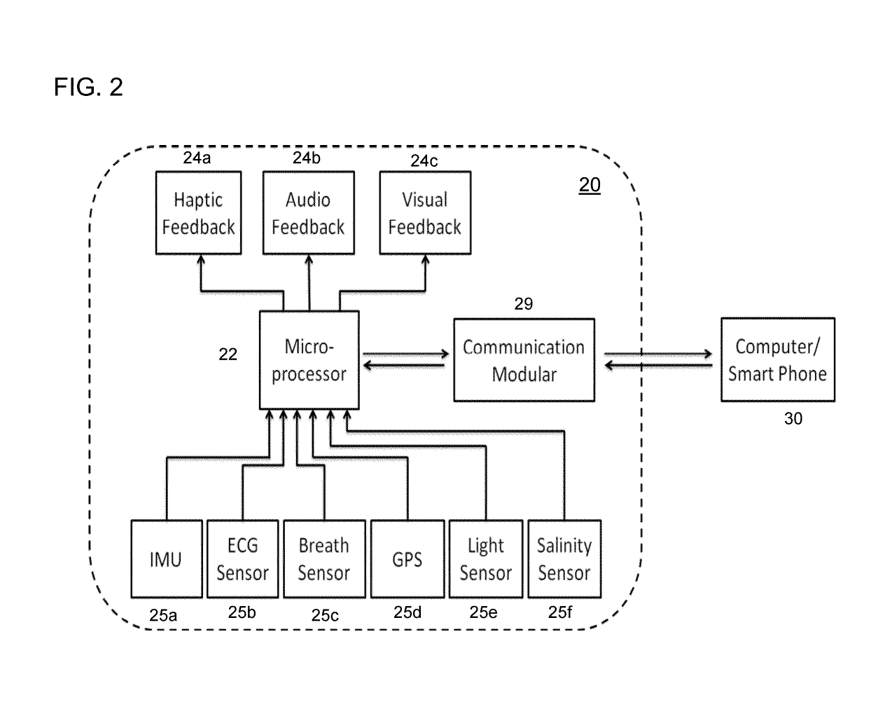

FIG. 2 is a block diagram depicting an example embodiment of a monitoring device 20 for integrating in a smart garment (e.g., the smart garment 10 or 200) and capable of communicating with a computer device 30. The monitoring device 20 includes a microprocessor 22, a communication modular 29, an inertial measurement unit (IMU) 25a, an ECG sensor 25b, a breath rate sensor 25c, a GPS 25d, a light sensor 25e, a salinity sensor 25f, a haptic feedback element 24a, an audio feedback element 24b, and a visual feedback element 24c. In some implementations, one or more of these components can be positioned on the computing device 30, for instance, the haptic feedback element 24a, the audio feedback element 24b, or the visual feedback element 24c.

In some embodiments, the IMU 25a is configured to measure and report (to the microprocessor 22) velocity, orientation, and/or gravitational forces. The IMU 25a can include accelerometers, gyroscopes, magnetometers, or a combination thereof.

The GPS 25d is configured to record locations coordinates of the wearer and provide the recorded coordinates to the microprocessor 22. The microprocessor 22 can use the received coordinates to calculate a trajectory of the wearer. The microprocessor 22 can compare the calculated trajectory to a pre-determined/stored path. Based on the comparison, the microprocessor 22 may cause the feedback elements 24a, 24b, and/or 24c to provide feedback to the wearer regarding a mismatch (or even a match) between the calculated trajectory and the pre-determined path. The microprocessor 22 may further provide the wearer through at least one of the feedback elements 24a-24c an orientation (or directions) to get back onto the pre-determined path. The microprocessor 22 can also calculate a distance traveled or an altitude climbed by the wearer based on the recorded coordinates.

The light sensor 25e is configured to measure ambient (or sun) light level and report the measured values to the microprocessor 22. The microprocessor 22 can use the measured values to estimate/calculate the wearer's exposure to sunlight. The monitoring device 20 may include other environmental sensors. For instance, pressure sensors can be integrated to measure atmospheric or water pressure. For mountaineers, atmospheric pressure is an important parameter to keep track of when climbing mountains. Also, water pressure is important for divers. Other environmental sensors that may be integrated in the monitoring device 20 include humidity sensors to measure air humidity or digital thermometers to measure environment temperature. Outside pressure and/or air humidity can have an impact on the wearer's breathing. Environment temperature and wearer's exposure to sunlight can affect the wearer's transpiration rate.

The salinity sensor 25f is configured to record salinity levels of the wearer's sweat. The salinity level can be used by the salinity sensor or the microprocessor to calculate/determine a dehydration level of the wearer. The dehydration level may be obtained using a lookup table or a formulation stored in the salinity sensor 25f or the microprocessor 22.

In some embodiments, the microprocessor 22, the IMU sensor 25a, the GPS 25d, the light sensor 25e, and the salinity sensor 25f are located (within the garment) at stable spot (in terms of motion, such as the top of the back along the wearers spine (the middle with respect to medial-lateral). In such embodiments, the recorded signals reflect mainly the upper body motion and include less of the shaking/jittery motion of the garment. The location at the top of the back (or top of the chest) is also convenient with respect to cell phone and GPS signal detection. Some portions/elements of the salinity sensor 25f may also be located around the chest in order to have more exposure to sweat.

The breath rate sensor 25c is configured to measure and report to the microprocessor 22 the change of the volumes by measuring the change in impedance of the conductive fabric. In some embodiments, impedance of a conductive fabric may vary based on the amount of tension or other forces applied on the garment. The breath rate sensor 25c can include two pieces that can be located around the chest cavity and around the abdominal cavity, respectively.

In some embodiments, the ECG sensor 25b can include multiple contact points around the chest to extract, amplify, and filter small bio-potential signals reflecting the electrical activity of the heart. In some embodiments, both the ECG sensor 25b and the breath rate sensor 25c are located around the chest cavity and/or around the abdominal cavity close to the heart and lungs, respectively.

In some embodiments, the sensors 25 (such as 25a-25f) can be used in combination to extract/calculate secondary information such as exertion levels, fatigue, dehydration, stress levels, metabolic consumption, relative movement of one body part to another, etc., and inform the wearer about the calculated values. The extraction/calculation of such secondary information can be performed by the microprocessor 22. For instance, heart rate and breathing volume can be used to detect exertion level. The microprocessor 22 can use salinity level of the sweat to calculate/estimate a dehydration level. The microprocessor 22 can also use measured skin conductivity to determine stress levels of the wearer. The microprocessor 22 can also use a combination of breath volume measurements and heart rate measurements to estimate consumption of oxygen by the wearer. Values of oxygen consumption can be used with heart rate measurements to estimate metabolic consumption. Also, the microprocessor can use distance traveled and altitude values (obtained based on recorded GPS coordinates) in combination with heart rate and breathing volume to determine an exertion level or amount of calories burned by the wearer.

In some embodiments, the microprocessor 22 can record measurements from different accelerometers associated with different parts of the wearer's body to perform motion analysis. For instance, the microprocessor 22 can track relative motion of upper and lower parts of an arm, relative motion of upper and lower parts of a leg, relative motion of two legs, relative motion two arms, or relative motion of arms and legs. The microprocessor 22 can generate a visual representation of a pattern depicting a body part motion. For professional (as well as amateur) athletes, such visual representation can help the athlete improve respective performance by understanding and perfecting limbs' movements.

The microprocessor 22 can use stored data (such as lookup tables, charts, etc.), mathematical formulations, statistical analysis, or a combination thereof to calculate the secondary information values.

In some embodiments, the microprocessor 22 is configured to use the data collected from the sensors 25, secondary information calculated based on collected sensor data, and/or analysis results (results of analyzing the collected data and/or the secondary information) to provide feedback to the wearer through one or more of the feedback elements 24a-24c. For instance, the microprocessor 22 may determine based on the collected data, secondary information, and/or any analysis results what message is to be conveyed to the wearer. In some embodiments, the message includes measured data, secondary information values, or analysis data to be displayed to the wearer, data indicative of current state of motion or state of physical well-being (e.g. heart rate too high/low, breathing volume too shallow, impact of landing during walking/running too high/too low). In some other embodiments, the message includes an evaluation of the wearer's performance during a physical exercise session. The microprocessor 22 may indicate to the whether a respective exercising performance is within a certain ideal or set range by presenting the data in a visual format after the training session is completed. The message may include instructions to the wearer, such as “slow down,” “go faster,” “drink water,” “take a deep breath,” “check blood pressure,” or the like. In other embodiments, the message can include a warning, such as “irregular heart beat,” “high dehydration level,” “slow breathing rate,” “severe atmospheric pressure,” or the like.

In some embodiments, the microprocessor 22 is configured to act responsive to the collected data in real time by triggering feedback element 24 to generate a signal to be provided to the wearer. The haptic feedback element 24a is configured, when triggered by the microprocessor, to generate a haptic gesturing (such as vibration or other mechanical stimuli). The haptic signal may be understood by the wearer to indicate a given message (such as wrong orientation/direction, slow motion, fast pace, or the like). Alternatively, the haptic signal may be generated to signal to the wearer to check the audio feedback element 24b and/or the visual feedback element 24c. The audio feedback element 24b is configured to produce audio signals such as a speech (e.g., indicative of instructions, performance data, or the like) or a non-speech signal (such a beeping sound, an alarm sound, or the like). The visual feedback element 24c is configured to generate visual signals. The visual feedback element 24c includes a display integrated on the smart garment 10 or a detachable display that can be worn similar to a watch. In some embodiments, the monitoring device 20 does not include a visual feedback element 24c. In such embodiments, visual data can be sent to the computer device 30 (such as a smart phone, a PC, a laptop, a tablet, a server, or the like) through the communication modular 29.

The visual data can then be accessed through the communication device 30. In some embodiments, the computer device 30 can include an application to perform some analysis data received from the monitoring device 20. For instance, an application running on a client device (such as a smart phone, tablet, PC, or laptop) can receive motion data and generate motion patterns of one or more limbs. The patterns can then be viewed by the wearer. The computer device 30 can also be configured to forward, via a communications network, at least part of the data received from the monitoring device 20 to a third party such as a healthcare provider, a physical trainer, a website, or the like.

A person of ordinary skill in the art should appreciate that different combinations of sensors and feedback elements can be employed in the monitoring device 20.

In some embodiments, the haptic feedback element 24a and the visual feedback element 24c are located on both sleeves of the smart garment 10. The symmetry property (on both sleeves) guarantees the quality of the signals communicated to the wearer and can give the wearer very clear and intuitive directions. In some embodiments, the feedback elements 24 are located in such a way that they are easily detected by the wearer and are located on the wearers body in such a way that the wearer can defer direction (e.g., using the bodies left-right and front-back symmetry).

In some embodiments, the data collected by the monitoring device 20 can be sent to a third party via uploading (through the communication modulator 29) the information to a server where the information can be accessed by the third party. The third party can be a healthcare provider of the wearer, a physical trainer of the wearer, or the like. The third party can then send recommendations or settings directly to the monitoring device 20 through a communication network and the communication modular 29. The recommendations/settings can then be indicated to the wearer of the monitoring device 20 either via haptic, audio, or visual output. Such recommendations/settings can include navigation information in order to upload a certain set of GPS coordinates or allow for a current path to be updated by the third party. Also information indicative of relative motion (of different body parts), or level of perspiration from an orthotic can be sent to and viewed by the third party. The third party can respond with instructions/recommendations to the wearer. In other embodiments, for a wearer with a chronic (or severe disease) some of the measured data (such as ECG measurements or breath rate measurements) can be sent to a healthcare provider on a regular or irregular basis.

In some embodiments, the motion tracking sensors can include components, such as gyroscopes (for example, component ITG-3200), and accelerometers (for example, component ADXL345). A global positioning sensor may include GPS components, such a GP35T or LS20031. An ECG sensor may include ECG sensing components, such as ALS-PT19-315C/L177/TR8.

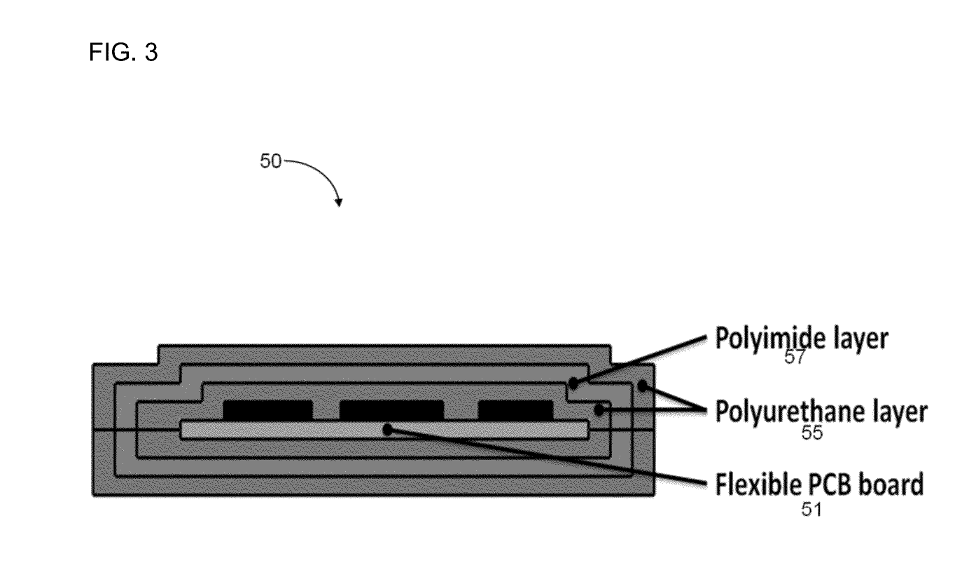

FIG. 3 is a diagram depicting a cross-sectional view of a printed circuit board 50 that can be integrated in a smart garment. The PCB 50 includes a flexible PCB 51, two polyurethane layers 55, and a polyimide layer 57. The components of the controller such as the controller 20, 100, 350 or any other controller described herein can be integrated on the flexible PCB 50.

To protect the electronics on the smart garment (such as the smart garment 10) from regular wash and static charge, special protection procedures can be implemented. Multi-layered heat sealable elastomeric adhesive film is extruded on the flexible PCB 51 to protect and adhere on the garment. The multi-layered adhesive film includes two polyurethane layers 55 and one polyimide layer 57. The two polyurethane layers 55 protect the flexible PCB 51 and the polyimide layer 57 from sweat and environmental factors. Any other adhesive film can also be used, for example silicone or Vulcan® rubber laminate.

The polyimide layer 57 is built to protect the electronics in the flexible PCB 51 from static charge. The laminate can be on both sides of the flexible PCB 51 creating an envelope to enclose the electronics. The films can be bonded to the flexible PCB 51 separately. A custom made jig can be used to press those films on to the flexible PCB 51. The films can also be pressed on other electronics (of the monitoring device 20) that are off the flexible PCB 51 (such feedback elements or some of the sensors). After sealing the flexible PCB 51 and other electronics of the monitoring device, the whole piece is attached to the garment by the same procedure. Furthermore, one or more sensors positioned on the smart garment (e.g., the sensor 15a-15f positioned on the smart garment 10) can also be laminated with the flexible protective films.

Wires (such as connecting wires 16 in FIG. 1) connecting sensors and feedback elements that are not on the main electronic board to the main electronic board can be implemented using coated conductive threads. The coated conducting threads can be sewn or laminated or otherwise integrated on or into the garment. The threads form into bigger conductive pattern at both ends to achieve a better and robust conductivity for the electronics. Those patterns are also protected by the heat sealable elastomeric adhesive film.

According to at least one aspect, integrating a monitoring device (such as the monitoring device 20) on clothing is more practical for many users who do not usually wear articles on their wrists. Also, for physical training purposes, sports garments with integrated monitoring device provide users with the ability to move and exercise freely while having performance measures accurately recorded without any extra burden on the users to carry any extra devices. Furthermore, having the sensors integrated on the garment allows for more physiometric data to be accurately collected (such ECG data, breath rate data, salinity data, and other data). The manufacturability is also a benefit as putting sensors in clothing can reduce the need to package the electronics into such a small form factor as is needed for wearing on the wrist and so can allow for longer battery life or otherwise more robust and powerful electronics and sensing units.

B. Computing and Network Environment

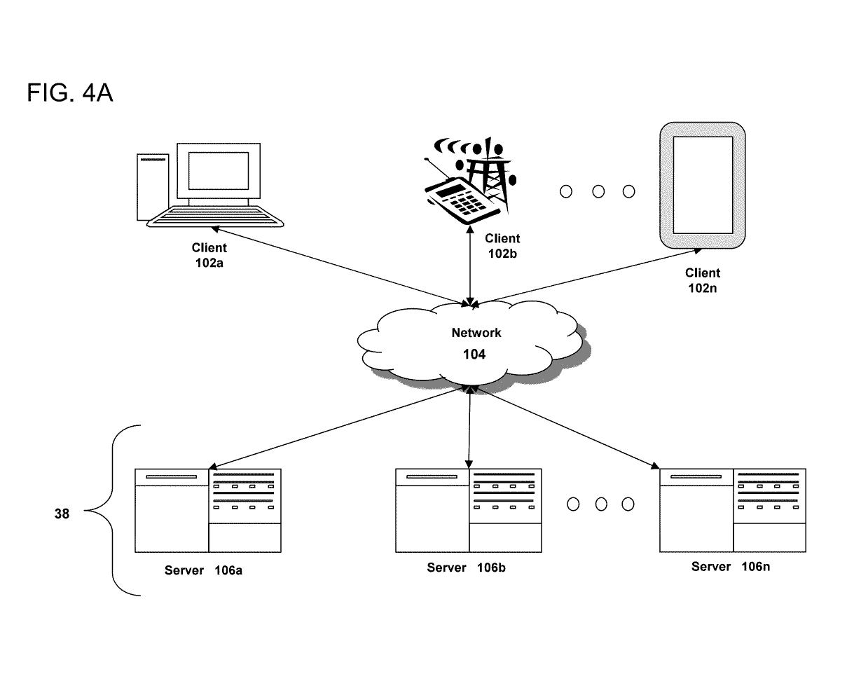

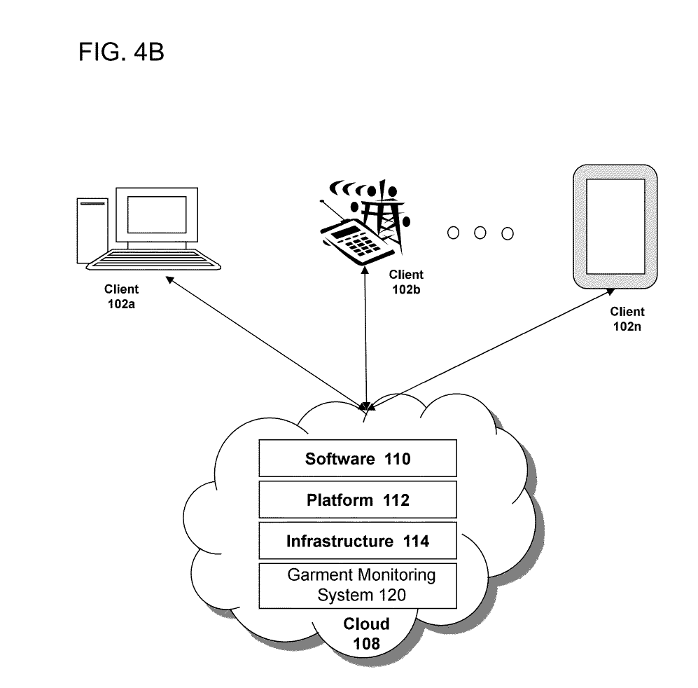

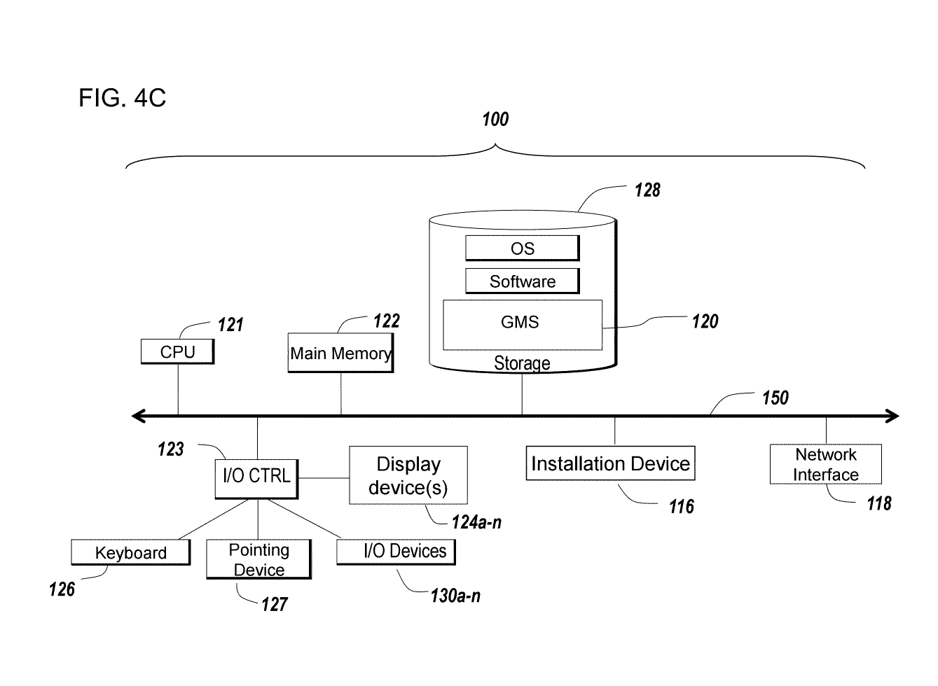

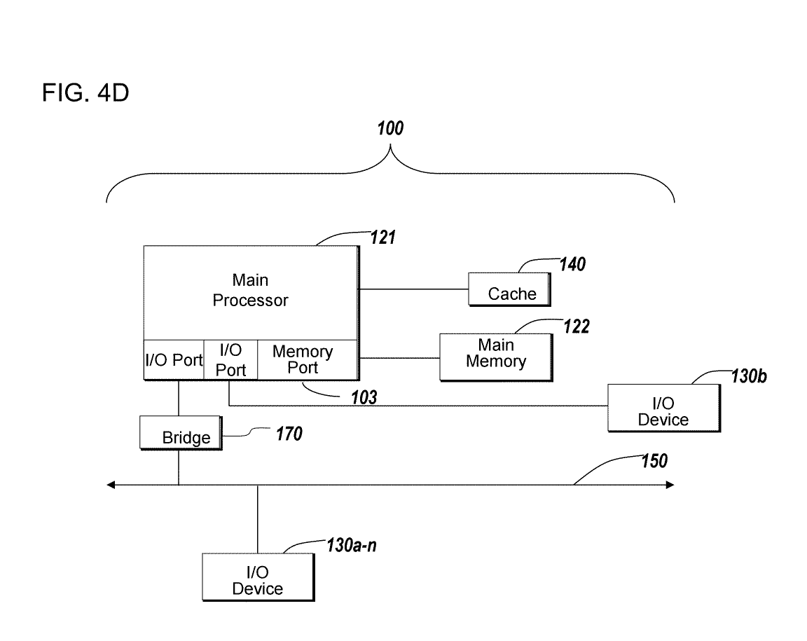

In addition to discussing specific embodiments of the present solution, it may be helpful to describe aspects of the operating environment as well as associated system components (e.g., hardware elements) in connection with the methods and systems described herein. Referring to FIG. 4A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 4A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 4B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS include AMAZON WEB SERVICES provided by Amazon.com, Inc., of Seattle, Wash., RACKSPACE CLOUD provided by Rackspace US, Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g. DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

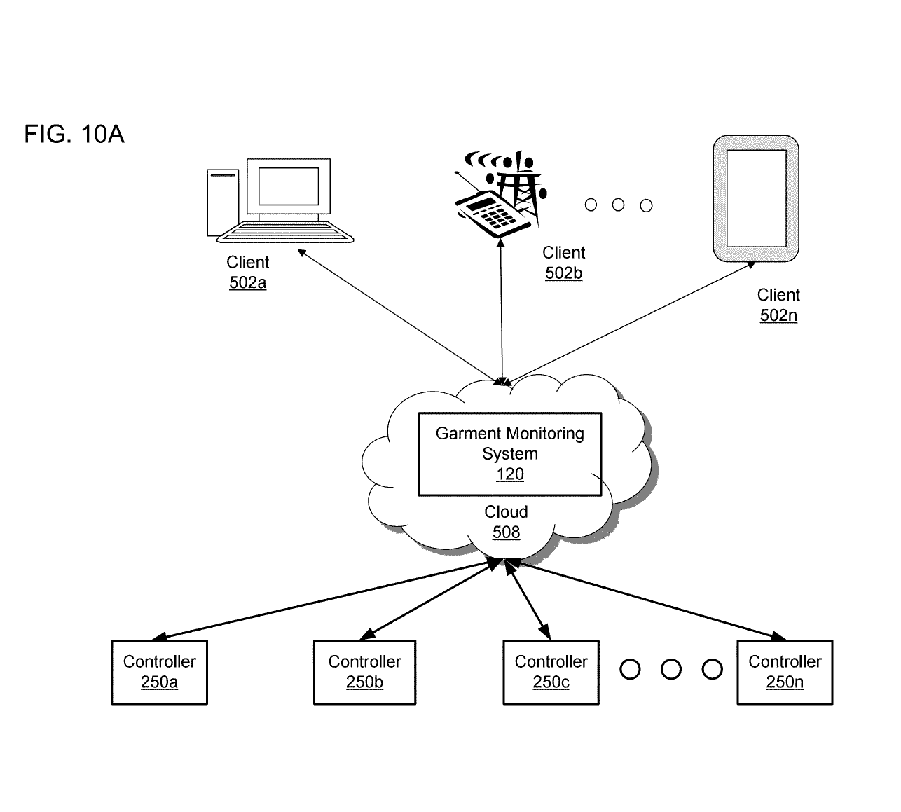

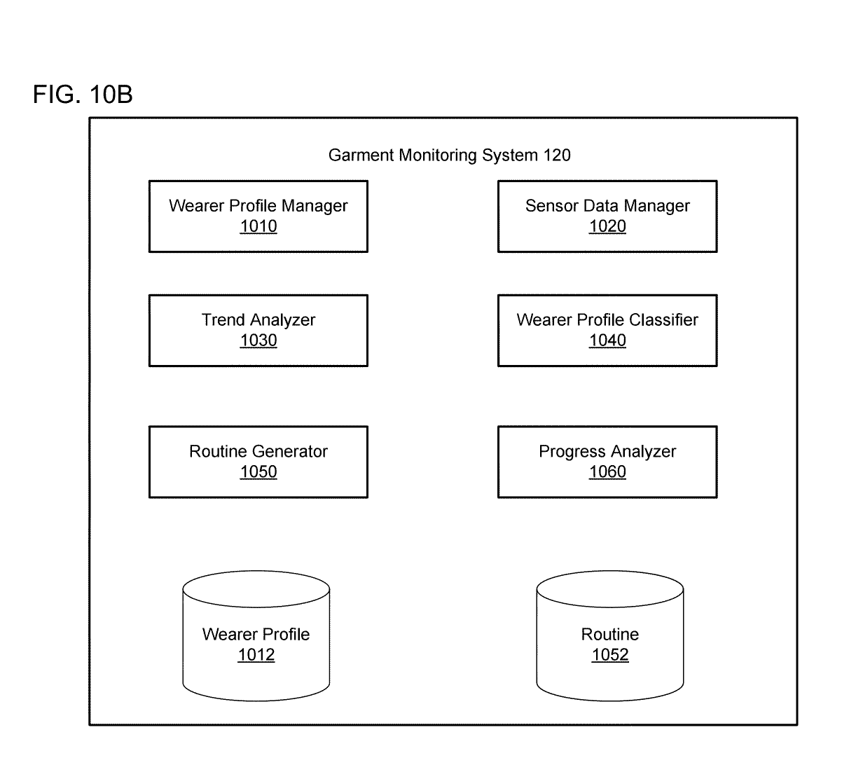

The cloud 108 may also include breathing rate analyzer 116. The cloud 108 can be communicatively coupled to a plurality of controllers (e.g., the cloud 508 connected to the plurality of controllers 550a-n shown in FIG. 10), each controller included in a smart garment that includes one or more sensors configured to measure a breathing rate of a user wearing the smart garment (e.g., the garment 10, 200 or any other smart garment described herein). In various embodiments, the smart garments can include resistance sensors which generate a resistance signal corresponding to a breathing rate or otherwise pattern of a user wearing the garment. The breathing rate signals, data or otherwise breathing pattern information is communicated to the cloud 108 via any of the wireless communication methodology described herein (e.g., directly to the cloud via a communications module included in a controller of the plurality of garments, or communicated to a client such as the clients 102a-n for communication to the cloud).

The breathing rate analyzer 116 may analyze the signal or data received from each of the plurality of controllers and determine a breathing pattern of each of the user. In various embodiments, such breathing data signal or data corresponds to the user performing a specific exercise routine, for example yoga, tai-chi, running, weight lifting, cross-fit or any other physical activity. In particular embodiments, the breathing rate analyzer can compare the breathing pattern of the user with reference breathing patterns to determine a qualitative and/or quantitative performance of a user. The breathing rate analyzer 116 can communicate feedback to the user, for example via any of the clients 102a-n described herein (e.g., a smartphone, tablet or smartwatch app, computer program, display provided on the garment, email communication, etc.).

The breathing rate analyze 116 can also provide instructions or suggestion to the user to improve the performance of the user based on the breathing pattern. In various embodiments, the breathing rate analyzer 116 can analyze and compare the breathing patterns of a plurality of users received from each of the plurality of controllers (e.g., the controllers 550a-n shown in FIG. 10. For example, the breathing rate analyzer can compare the breathing pattern of the plurality of users to rate the performance of a user relative to other users, for example develop a quantitative or qualitative rank. This creates a game environment among the plurality of users to encourage performance improvement. The cloud 108 can provide user incentives such as app level upgrades, commendations, elevation leaderboard rankings, discounts, rebates or any other incentives to encourage the users to improve their performance.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, Calif.). Clients 102 may also access SaaS resources through smartphone or tablet applications, including, for example, Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 4C and 4D depict block diagrams of a computing device 100 useful for practicing an embodiment of the client 102 or a server 106. As shown in FIGS. 4C and 4D, each computing device 100 includes a central processing unit 121, and a main memory unit 122. As shown in FIG. 4C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a garment monitoring system (GMS) 120. As shown in FIG. 4D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of a multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 4C, the processor 121 communicates with main memory 122 via a system bus 150 (described in more detail below). FIG. 4D depicts an embodiment of a computing device 100 in which the processor communicates directly with main memory 122 via a memory port 103. For example, in FIG. 4D the main memory 122 may be DRDRAM.

FIG. 4D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 4D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 4D depicts an embodiment of a computer 100 in which the main processor 121 communicates directly with I/O device 130b or other processors 121′ via HYPERTRANSPORT, RAPIDIO, or INFINIBAND communications technology. FIG. 4D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 4C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 4C, the computing device 100 may comprise a storage device 128 (e.g. one or more hard disk drives or redundant arrays of independent disks) for storing an operating system or other related software, and for storing application software programs such as any program related to the software 120 for the garment monitoring system. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via a I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 4B and 4C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, RIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Wash. In other embodiments, the computing device 100 is a eBook reader, e.g. the KINDLE family of devices by Amazon.com, or NOOK family of devices by Barnes & Noble, Inc. of New York City, N.Y.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 is monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

C. A Garment that Includes a Pair of Sensing Components Integrated into the Garment and Configured to Electrically Couple to a Controller

Various embodiments of the systems and methods described herein relate to a garment that includes one or more sensing components strategically integrated onto the garment. The garment can be an item of clothing wearable by a wearer. In some implementations, the garment can be a shirt, shorts, belt, and a wrap, a band, such as a wristband, an arm band, a waistband or a headband, among others. In some implementations, the garment can be any fabric or material that includes sensing components that can measure or sense changes to one or more physical changes occurring within a user, such as a wearer. The physical changes can include an expansion or contraction of a muscle, the extension or contraction of a joint, or movement of skin, muscle, joints, among others. In some implementations, the garment can include one or more strategically positioned resistance-based sensors. In some embodiments, the garment can include or otherwise couple to one or more position or motion detection sensors configured to detect motion, changes in position or posture, among others. In some embodiments, the garment can be configured to couple to or otherwise communicate with a controller that can communicate with one or more sensors integrated into the garment or otherwise sensing physical changes of the wearer of the garment.

In one embodiment, the garment can be a shirt designed to fit closely around a torso of the wearer. The shirt can be made of a stretchable fabric and sized and shaped to be in contact with a majority portion of the wearer's torso. For instance, the shirt can be a compression shirt. In some implementations, the shirt can include a portion that is made from a stretchable fabric. The shirt can include one or more sensing components integrated into the shirt. The sensing components can include a stretchable resistance based sensor that is configured to change in resistance based on a length of the sensor, or in other words, change in resistance based on a force applied to the sensor that causes the sensor to extend from a first length when the sensor is in a relaxed state to a second length greater than the first length when the sensor is in a state in which a force is applied to it. In some implementations, the sensor can be configured such that it has a first stretchability along a first axis and a second stretchability along a second axis. In some implementations the sensor can be shaped to have a first length along the first axis and a second length that is longer than the first length along the second axis. The sensing component can be configured to change in length responsive to a wearer's breathing. When the wearer inhales, the chest cavity expands due to the air inside the cavity causing the circumference of the chest cavity to increase. Conversely, when the wearer exhales, the chest cavity contracts as air leaves the cavity causing the circumference of the chest cavity to decrease. The sensing component integrated into the garment can be strategically located and oriented such that the sensing component extends in length when the wearer inhales as the garment around the torso stretches to accommodate the expanded chest cavity and conversely, the sensing component contracts in length when the wearer exhales causing the garment to return to its relaxed state.

As will be described herein, the resistance values measured across the sensing component can be used to determine various metrics of the wearer. For instance, the resistance values can be used to determine a breathing pattern of the wearer, a breathing volume of the wearer, a breathing rate of the wearer, a breathing capacity of a wearer, among others. Further, other types of sensors can be integrated into the garment that may detect electrical signals generated by the heart or through muscle expansion and contraction, among others. These sensors can be used to measure a heart rate, among others, which in conjunction with the breathing data, can be used to determine or identify one or more conditions of the wearer.

In some embodiments, the garment can be configured to include or otherwise couple to a position or motion sensor, such as a gyroscope or accelerometer. Readings from these sensors can be used to determine a wearer's posture, stability, strength, fatigue threshold, flexibility, among others. This in turn can be used to determine whether a wearer is performing an exercise as desired, measure progress over time of the wearer's performance. For instance, the shirt can be used by a yoga instructor or student. In such a use case, the sensing components can be used to determine types of activities performed by the user, a number of times the activities were performed, the breathing patterns of the wearer as the activities were performed, the stability of the wearer as each activity was performed, among others. The data measured by a controller coupled to each of these sensing components can then be used to determine progress over time.

In some embodiments, the data measured by the controller can be transmitted to a server that collects data from a plurality of controllers corresponding to garments worn by different wearers. The data can be aggregated and used to establish trends, compare a wearer to other wearers, and identify wearers that may be similar to one another based on their performance and measured values.

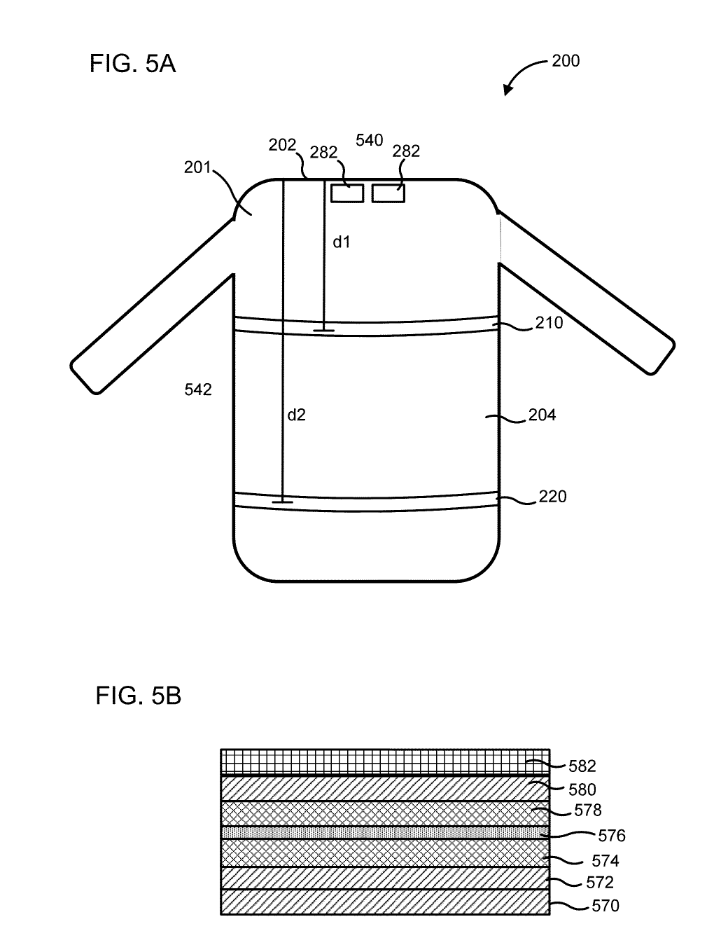

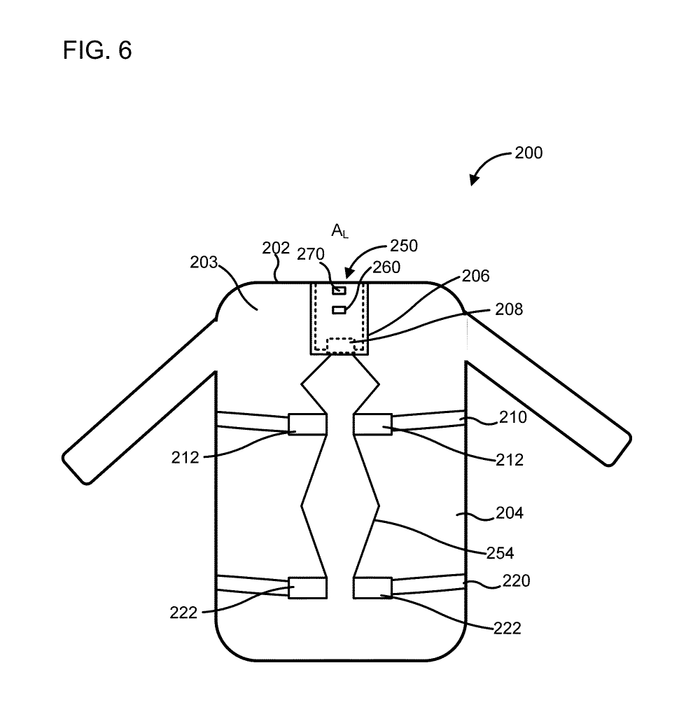

Referring now to FIGS. 5A and 6 show an embodiment of a garment 200 configured to measure one or more parameters of a wearer. As shown in FIG. 5A, the garment is a shirt. The shirt can include a base material or fabric. In some implementations, the base material 204 can be formed from any material suitable to be worn. In some implementations, the base material can be a stretchable material, such as nylon, polyester, cotton or a blend of one or more materials. In some implementations, the shirt can be a compression shirt configured to fit a wearer snugly. In some embodiments, the base material can be made from a stretchable material that conforms to a shape of a wearer.

As shown in FIG. 5A, the shirt includes a front side 201 of the garment configured to cover a front portion of a wearer. FIG. 6 shows a back side 203 of the shirt 200. Although the garment depicted in FIGS. 5A and 6 is a shirt, the garment can be any type of garment that can include a sensing component capable of measuring physical changes occurring within the wearer. For instance, the physical changes can include muscle expansion and contraction, bone movement, joint movement, among others. In some implementations, the changes may be non-physical. For instance, the sensing components may be capable of measuring temperature changes, electrical changes, among others occurring within the wearer's body or on a skin of a wearer. As shown in FIGS. 5A and 6, the shirt 200 includes a torso portion configured to surround a torso of a wearer. The garment can be shaped and sized to be any other type of garment, including but not limited to a vest, bra, waistband, wrap, or other garment worn by a wearer. In other embodiments, the base material 204 can be shaped and sized to be worn on any other portion of the body of a wearer, for example, shorts, a pant, a head brace, a belt, a patch, among others.

In some embodiments, a first sensing component 210 can be integrated into a first location of the base material 204 of the garment 200 corresponding to a predetermined region of the wearer. Furthermore, a second sensing component 220 can be integrated on a second location of the base material 204 different from the first location corresponding to another predetermined location of the wearer. The first and second sensing components 210 and 220 can have a first elastic stretchability along a first axis 540 of the base material 204, and a second elastic stretchability along a second axis 542 of the base material 204. The second elastic stretchability can be greater than the first elastic stretchability. The elastic stretchability of a material along an axis relates to an increase in length of the material per unit force. As such, if the same force was applied along both a first axis 540 and a second axis 542 of the sensing components 210 and 220, the length of the sensing components along the second axis 542 would increase more than the length along the first axis 540. In some implementations, the elastic stretchability of a material along an axis can be based on the dimensions of the material. A material that has a first length along a first axis and a second length that is greater than the first length along a second axis will have be more stretchable along the second axis, indicating a greater elastic stretchability.

In various embodiments, the first sensing component 210 and the second sensing component 220 can be electrically conductive. The sensing components 210 can be designed, constructed or configured such that the electrical resistance of the sensing components 210 and 220 change with a change in length. In some implementations, the sensing components 210 and 220 can be designed or constructed such that the electrical resistance of the sensing components increase as the length of the sensing components increase. By calibrating the sensing components, it is possible to determine a change in length of the sensing components based on a change in resistance. The change in length of the sensing components can be used to detect breathing patterns of a wearer if the base material on which the sensing components are integrated changes in length as the wearer breathes in and out. Additional details relating to the functionality of the sensing components 210 and 220 are provided herein.

FIG. 5B shows a side cross-section of a portion of a garment that includes the first sensing component 210 according to a particular embodiment. The second sensing component 220 can be substantially similar to the first sensing component 210 in structure and function. In various embodiments, the first sensing component 210 can be integrated into the base material 570. The first sensing component 210 can include one or more layers.

In some implementations, the first sensing component 210 can be integrated into the base material 570 of a garment. In some implementations, to integrate the sensing component 210 to the base material 570, a first layer 572 that can include a film, such as a plastic film, is applied to the base material. The plastic film may be sewn, heat pressed, or otherwise integrated to the base material. A first layer of a material 574 with low water solubility may be applied to an upper surface of the plastic film 572.

A conductive layer 576 is then formed on the layer of material with low water solubility. The conductive layer 576 can be an electrically conductive layer that can include electrically conductive material configured to conduct electrical current through the conductive layer 576. The electrically conductive material can include electrically conductive particles deposited, coated or otherwise positioned on top of the first layer of material 574 with low water solubility. In some implementations, the electrically conductive layer 576 can include a strip, wire, thread, or other electrically conductive material.

A second layer of material 578 with low water solubility is formed on top of the conductive layer 576 such that the conductive layer is encapsulated by the material with low water solubility. In some implementations, a second layer of plastic film 580 is formed on top of the second layer of low water solubility 578. Another fabric 582 can then be applied or otherwise attached to the plastic film 580.

To prevent the conductive layer 576 from being adversely affected by water, the layers adjacent to the conductive layer 576 can be made from a material that has a low water solubility. In some implementations, the material can have a water solubility below a predetermined threshold, for instance, below 600 μg/100 g at 50° C. In some implementations, the material can include silver chloride. By coating the conductive layer 576 with a material or compound that has low water solubility, the conductive layer 576 can be protected from water, thereby increasing the number of washes the garment can withstand before the sensing component is exposed.

It should be appreciated that one or more of the layers may not be included in the sensing component 210. For instance, the sensing component may include one or more of the layers 572-582. In some implementations, the sensing component may include one or more layers 572-582 and the conductive layer 576.

In some implementations, the layers 574 and 578 can be made from an electrically inert material to isolate any electrical charges carried by the electrically conductive second layer 576. In some implementations, the conductive layer 576 can be plated onto one or more underlying layers, for example, using a roll-to-roll chemical plating technique, drop coated or spray coated with the conductive material to evenly deposit the conductive material on the underlying layers 572 and 574. The conductive material can include, for example metallic particles or other electrically conductive materials. Examples of conductive material can include carbon nanotubes, gold nanoparticles, conductive polymer ink, for instance, poly(3,4-ethylenedioxythiophene (PEDOT:PSS), silver/silver chloride ink, gold ink, among others. In other embodiments, the conductive layer 576 can include conductive wires, threads, or other objects that may wrap, surround, intertwine, weave or otherwise be in contact with the underlying layers 572 and 574. Examples of such materials can include copper yarn or steel wool filament woven into the first layer.

In some embodiments, a nylon-polyester, “spandex”, fabric that is relatively non-elastic in the weft and elastic in the warp can be used as the first layer 212. In some implementations, the weft can be made using a first plurality of threads, while the warp can be made using a second plurality of threads having a greater elastic stretchability. The underlying layers 572 and 574 can be plated using a roll-to-roll chemical plating technique that deposits electrically conductive particles, such as silver atoms evenly on the underlying layers 572 and 574. By coating the underlying layers 572 and 574 with silver enables a resistance change to be measured when the sensing component 210 is stretched along the warp. As will be described herein, the sensing components 210 and 220 can be integrated into locations of the shirt such that the sensing components are able to detect electrical resistance changes when the wearer breathes in and out. The sensing components can be positioned around the circumference of a wearer's torso, and as the wearer breathes causing the shirt and the first layer integrated into the shirt to change lengths during the course of a breathing routine, a change in resistance corresponding to the expansion and contraction of the person's torso cavity can be measured. It should be appreciated that the elastic properties of the first layer are important as the resistance change can be a function of the elastic properties of the first layer. If the first layer 212 were to stretch in both directions (the weft and the warp), determining whether the change in resistance can be attributed to a change in length along the weft or the warp would be difficult. A change in length along the weft can be attributed to a wearer stretching vertically, while a change in length along the warp can be attributed to circumferential elongation due to breathing Accordingly, it is desirable to reduce the amount of stretching along the weft such that any resistance change detected can be attributed to a change in length of the first layer along the warp. By confining the stretch direction to a single axis (the warp), any change in resistance can be attributed to circumferential elongation or contraction. In this way, a system utilizing the resistance change can manipulate the resulting signal to filter unwanted noise and motion artifacts and accurately determine breathing patterns. It should be appreciated that the warp and weft correspond to threads in a fabric. A fabric that is more stretchable along the weft relative to the warp may also be used. The important point to note is that the more stretchable axis of the fabric should be aligned with an axis along which the elongation happens. In the case of the wearer's chest, the more stretchable axis of the fabric should be aligned with an axis extending along the width of the chest muscle to isolate resistance change in the sensing component to changes in length in the circumference of the wearer's chest.

In some implementations, the layers 572 and 580 can include an adhesive protective material, such as thermal poly urethane (TPU). The layers 572 and 580 can be heat and/or pressure activated. The sensing component including one or more of the layers 572-582 can be laminated with one or more layers of TPU film. Laminating the sensing component can provide numerous advantages including allowing the layer 572 with the electrically conductive material 576 thereon to be seamlessly manufactured into the underlying base component 570 in a robust and machine washable way to form the first sensing component 210 and the second sensing component 220. Another advantage includes structurally stabilizing the second plurality of threads forming base layer 570 by impregnating the second plurality of threads as well as the conductive material 576 thereon with the adhesive which makes them less prone to producing electrical noise that could result from individual threads “sliding” resulting in a resistance change. Another advantage includes providing the first sensing component 210 and the second sensing component 220 with additional protection against outside elements water, sweat, wear and tear in addition to a silver chloride coating positioned thereon. It should be understandable that while the first sensing component 210 and the second sensing component 230 are shown as positioned on an outside surface of the garment 200 so that they are visible, in other embodiments, the first sensing component 210 and the second sensing component 230 can be positioned on an inner surface of the garment 200 so that they are not externally visible. In various embodiments, the sensing component can be attached to the base material 570 after the multiple layers that form the sensing component have been deposited. In some implementations, the sensing component may not include one or more of the plastic film layers 572 and 580, one or more of the layers 574 and 576, or the layer 582.

To form the first sensing component 210 and the second sensing component 220 to permanently secure to the base material 204 of the garment, the sensing components 210 and 220 can be cut in longitudinal strips having any suitable width. It has been determined that the elastic stretchability of a material along a first axis is inversely proportional to a length of the material along a second axis substantially perpendicular to the first axis. As such, to increase the elastic stretchability of a material along the first axis, it may be desirable to reduce the length of the material along the second axis. In an effort to generate to isolate changes in resistance of the sensing component to changes in lengths along one axis, having a shorter length in the second axis is desirable. Accordingly, the width of the longitudinal strips may be kept to a width less than a predetermined threshold. In some implementations, the width can range from 0.1 mm to 5 cm, from 0.5 mm to 1 cm, from 0.5 mm to 2 mm, and so forth. It is possible to have widths greater than 5 cm. The length of the sensing components can be varied to accommodate various sizes of the garments on which the sensing components are to be integrated.

As shown in FIGS. 5A and 6, the first sensing component 210 and the second sensing component 220 can include strips positioned circumferentially around a torso region of the shirt. The strips can be integrated such that the strips have a first elastic stretchability along a weft of the base material 204 and a second elastic stretchability greater than the first elastic stretchability along a warp of the base material 204. In this way, when the circumference of the wearer's torso increases or decreases, the strips can experience a change in electrical resistance. It should be appreciated that a size of the change in resistance can be mapped to a change in length in circumference. In this way, based on the size of the resistance change, a controller coupled to the sensing components can determine a size of the change in the length of the circumference.

For instance, when a wearer breathes, the wearer's chest can go through an expansion and contraction. During a breathing cycle, a wearer can begin to inhale, causing air to enter their lungs, thereby expanding the circumference of their chest area. When the wearer begins to exhale, air is expelled from the lungs causing the circumference of the chest area to decrease until the wearer starts to inhale again. It should be appreciated that the circumference of the wearer's chest is at its maximum when the wearer has fully inhaled, while the circumference of the wearer's chest is at its minimum when the wearer has fully exhaled. By knowing the maximum and minimum circumferences, it is possible to track a wearer's breathing by monitoring the circumference of the wearer's chest. The present disclosure utilizes resistance based sensing components to track a wearer's breathing by monitoring a length of the circumference of the wearer over time. The present disclosure describes sensing components that can be used to determine a length of a circumference of a wearer based on a change in resistance of the sensing component. A shirt that conforms to the wearer's body can be configured to stretch as the wearer inhales and relax when the wearer exhales. The sensing components described herein can be designed, constructed or configured to be integrated into the shirt such that the sensing component also stretches when the wearer inhales and relaxes when the wearer exhales. Further, the sensing components can include a conductive material that has a resistance value that changes as the length of the sensing component changes. In this way, when the sensing component is stretched as the wearer inhales, the resistance value of the sensing component can increase as the wearer's chest expands. When the circumference of the wearer's chest is at a maximum, the sensing component's resistance value may also be at a maximum as the length of the sensing component will be at a maximum. Conversely, when the circumference of the wearer's chest is at a minimum, the sensing component's resistance value may also be at a minimum as the length of the sensing component will be at a minimum. The maximum resistance value and the minimum resistance value can be identified and used as data points for calibrating the sensing component. By identifying the maximum and minimum resistance values and the corresponding lengths of the sensing components, a controller can determine a length of the sensing component (which is correlated to the circumference of the chest) based on the resistance value across the sensing component. In this way, by monitoring the resistance across the sensing component over time while the wearer is wearing the garment, the controller can map out the wearer's breathing by correlating the resistance value of the sensing component to lengths of the sensing component and the circumference of the chest area, which is indicative of the user's breathing.

As shown in FIGS. 5A and 6, the first sensing component 210 can be integrated into the shirt 200 at a first location that is a first distance d1 from a top edge 202 or neckline 202 of the shirt 200 such that the first location corresponds to a pectoral region of the wearer when the wearer wears the shirt. In some implementations, the sensing component 210 is positioned on the base material 204 such that the sensing component is positioned between the lowest rib and a plane extending from one armpit of the wearer to the other armpit of the wearer. The first sensing component 210 may be used to measure the contraction and expansion of the rib cage and chest cavity during breathing.

The second sensing component 220 can be integrated into the shirt 200 at a second location, which can be a second distance d2 from the neckline 202 such that the second location corresponds to an abdominal region of the wearer. In some implementations, the second location can be between the lowest rib and a region between the pelvis and belly button of the wearer second sensing component when the wearer wears the shirt. In this manner, the first sensing component 210 expands or contracts in response to chest expansion/contraction, and the second sensing component 220 expands or contracts in response to abdominal expansion/contraction, respectively. The expansion and contraction of the chest and abdomen of a wearer can be used to determine breathing patterns, rate and quality of the wearer, among others. The second sensing component 220 can be used to measure the contraction and expansion of the abdominal cavity as it occurs independent of the chest cavity.

It is to be noted that separate measurements of chest expansion and contraction as well as abdomen expansion and contraction are particularly useful in wearers performing Yoga in which places particular emphasis on diaphragmatic or chest breathing vs abdominal breathing. Positioning of the first sensing component 210 around the pectoral region and the second sensing component 220 around the abdominal region provides information on breathing form of the wearer which is very useful in determining the performance of the wearer in maintaining or otherwise performing various Yoga poses, as described in further detail herein. However, the breathing rate or patterns or any other physiometric parameters determined using the garment 200 can be useful for determining the performance of the wearer performing any physical activity such as tai-chi, running, weight lifting, cross-fit, wrestling, tennis, football, soccer, cricket or any other physical activity.

In other embodiments, one or more of the first sensing component 210 and the second sensing component 220 can be configured to be positioned around a muscle of the wearer to detect and measure the expansion and contraction of the muscle. For example, the first sensing component 210 and/or the second sensing component 220 can be configured to be positioned on a bicep, a tricep, an abdominal muscle, a thigh muscle or any other muscle of the wearer wearing the garment 200. The first sensing component 210 and/or the second sensing component 220 can be used to measure an expansion or contraction of the muscle, for example to determine muscle engagement, movement, activity, strength, among others. This is particularly beneficial for determining muscle degeneration and/or regeneration and activity of immobilized wearers, for example observing paralyzed wearers or wearers undergoing rehabilitation after an accident, stroke etc. to determine muscular degeneration, regeneration, fracture healing, overall health and progress of the wearer. Similarly, one or more of the first sensing component 210 and the second sensing component 220 can be configured to be positioned around a joint of the wearer, for instance, a knee joint, an elbow joint, a hip joint or any other bone joint to determine various metrics associated with the joints. For instance, the sensing components can be used to determine an amount of bend or extension in a particular joint. This may be helpful for patients suffering from ailments of the joints, such as arthritis, or recovering from an injury to a joint.

Referring to FIG. 6, the first sensing component 210 includes first electrical terminals 212 and the second sensing component 220 includes second electrical terminals 222 integrated into the back portion 203 of the garment 200. In various embodiments, the first electrical terminals 212 and the second electrical terminals 222 can include signal amplifiers, a resistance measuring circuit (e.g., a wheatstone bridge circuit) or any other suitable sensing component. The first sensing component 210 can include at least one electrical wire. The electrical wire can be part of the resistance measuring circuit and can be used to deliver a voltage across the first sensing component 210. The electrical wire can be configured to couple with a controller 250 that may or may not be a part of the garment. In some implementations, the resistance measuring circuit can be configured to allow the controller to measure an electrical resistance across at least a portion of the first sensing component 210. The resistance measuring circuit can include a voltage source configured to provide a voltage to the first sensing component and an ohmmeter or other resistance measurement component configured to sense or otherwise measure an electrical resistance of a portion of the first sensing component or the entire first sensing component. As described above, the resistance measurement component can determine an electrical resistance across the first sensing component and based on the electrical resistance across the first sensing component, the controller or a garment monitoring system, such as the GMS 120 shown in FIG. 4C can determine a change in length of the first sensing component 210 and correlate the change in length to a breathing parameter of the wearer. The second sensing component 220 can include similar components as the first sensing component 210.

In various embodiments, the wires 254 can be formed from a material which does not experience a change in resistance due to stretching or contracting or are not stretchable (e.g., metal such as copper, silver, gold, or aluminum wires). In this manner, the wires 254 have no influence on the resistance measurements made by the first sensing component 210 and the second first sensing component 220. In other embodiments, the wires 254 are formed from a stretchable material but are positioned proximate to a spinal cord of a wearer which negligibly stretches due to the chest or abdominal expansion and contraction. In various embodiments, the first sensing component 210 and the second sensing component 220 can be communicatively coupled to the controller 250 via a wireless connection, for example a Bluetooth®, low powered Bluetooth®, Wi-Fi, NFC or any other wireless connection.

The controller 250 has a compact form factor and is configured to be positioned in a compartment 206 defined on or within the garment 200. The compartment 206, for example, a sleeve or pocket can be positioned on the back portion 203 of the garment 200. In various embodiments, the compartment 206 can be sized to receive a device that includes the controller 250 and a housing configured to protect the controller 250 from water, sweat or moisture, among other elements that may adversely affect the functioning of the controller. In various embodiments, the controller 250 can include a processor and a memory storing computer-executable instructions. The device can further include a power source such as, a rechargeable battery, a kinetic battery or a solar cell for providing electrical power to the controller or the one or more sensing components positioned on the garment 200. The garment can include an attachment mechanism to secure the device to the garment 200 and electrically couple the controller 250 to an electrical port 208 that is positioned on the garment 200 and is electrically coupled to the one or more wires 254 of the first sensing component 210 and the second sensing component 220.

The device can further include one or more additional sensors. The sensors may be body orientation detection sensors. For example, FIG. 6 shows a first sensor 260 and a second sensor 270 included in the device. In various embodiments, the first sensor 260 can include at least one accelerometer (e.g., 3-axis digital accelerometer (e.g., ADXL362 from Analog Devices, Inc.) configured to sense acceleration data and the second sensor 270 can include a gyroscope, for example a digital 3-axis gyroscope (e.g., L3GD20 from STMicroelectronics) configured to determine spatial orientation data. In some implementations, the device can include a magnetometer. The acceleration data and the spatial orientation data can be used to determine a posture, orientation and/or activity of the wearer. In some implementations, the data generated from these sensors can be compared to predefined data ranges to determine, for example, a posture or orientation of the wearer, a stability of the wearer, among others. This can be useful for wearers performing yoga to determine which postures or orientations they were in and their stability while performing such postures, among others. Wearers performing other physical activities, such as, tai-chi, weight lifting, stretching or any other physical activity may find the information generated by the sensors 260 and 270 helpful. In various embodiments, the first sensor 260 and the second sensor 270 can be integrated with the garment 200, for example laminated onto the base material 204 via one or more layers of adhesive similar to the first sensing component 210 and the second sensing component 220.

In various embodiments, the first sensor 260 (e.g., an accelerometer) and the second sensor 270 (e.g., a gyroscope) can also be used for recognizing a space, environment or location of the wearer, determine a force of a foot of the wearer striking on the ground (e.g., during running), determining energy consumption of movements (e.g., calories burned), etc. In other embodiments, the first sensor 260 and the second sensor 270 can be positioned at a different location, for example a different garment or accessory worn by the wearer. For example, the first sensor 260 and/or the second sensor 270 can be positioned in a shoe of the wearer and configured to determine a posture, stability, impact absorption of a shoe, a stride, or any other physiometric or biometric parameter of the wearer. In such embodiments, the first sensor 260 and the second sensor 270 can wirelessly communicate with the controller 250 via any suitable wireless connection described herein (e.g., Bluetooth®, RFID, NFC, etc.). The controller 250 is configured to sample values from the first sensor 260 (e.g., an accelerometer) and the second sensor 270 (e.g., a gyroscope) at a predetermined frequency. The predetermined frequency can be varied based on the amount of exertion or movement of the wearer. For example, during fast or frequent movements, for example, during running, changing a yoga pose, wavering while maintaining a yoga posture, exercising etc., a fast frequency and thereby, sampling rate is increased. On the other hand, a slow sampling frequency can be used during slow or negligible movement, for example sitting, maintaining a posture, slow walking etc. In this way, an amount of data stored on a memory of the controller 250, as described herein can be minimized.

In various embodiments, the attachment mechanism is positioned on the back portion of the garment such that when the device including the controller is coupled to the attachment mechanism, the device including the sensors 260 and 270 is aligned with a spinal column of the wearer when the garment 200 is worn by the wearer. In some implementations in which the garment 200 is formed from a stretchable material, stretching of the garment 200 can result in displacement of the first sensor 260 and the second sensor 270 which can result in false signals or noise from these sensors. The portion of the garment 200 which is positioned proximate to the spinal cord and particularly the portion of the spinal cord located near the top edge 202 (i.e., proximate to the neckline 202 on the back portion 203) however, is expected to experience the least involuntary displacement or stretching, as this portion of the wearers body is physiologically isolated from chest and/or abdomen expansion/contraction during breathing. By positioning the controller 250 and thereby, the first sensor 260 and the second sensor 270 aligned with the spinal cord, for example located near the top edge 202 minimizes involuntary displacement of the first sensor 260 and the second sensor 270, thereby minimizing noise and false signals. Moreover, the location is convenient for a user to stretch their arm behind their head to attach and detach the device from the garment.

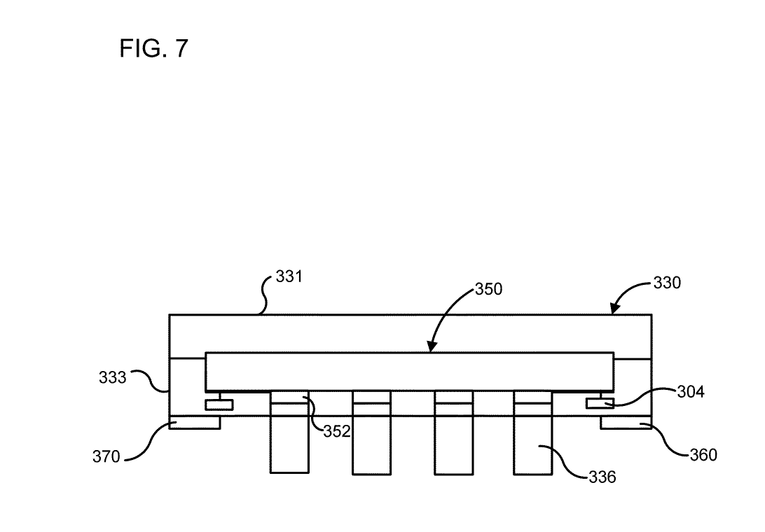

As described before, the controller 250 can be positioned within a housing, for example to protect the controller 250 from humidity, sweat and moisture. For example, FIG. 7 shows a controller 350 positioned within an internal volume defined by a housing 330. The controller 350 can be substantially similar to the controller 250 described herein. The housing 330 includes a first portion 331 and a second portion 333 which are coupled together to define an internal volume therebetween within which the controller 350 is positioned. A plurality of suspensions 304 (e.g., springs or compliance members such as foam pads, rubber pads, silicone pads, etc.) can be positioned within the internal volume for absorbing any shock on the controller 350 due to rapid movements of the wearer. Furthermore, at least one first sensor 360 (e.g., an accelerometer) and at least one second sensor 370 (e.g., a gyroscope) can be positioned on an outer surface of the housing 330 and communicatively coupled to the controller 350 positioned therewithin.

In various embodiments, the housing 330 includes a set of housing electrical connectors 336 configured to be coupled to a corresponding set of controller electrical couplings 352. In various embodiments, the housing electrical connectors 336 can include contact couplings, mechanical couplings, snap-fit couplings or any other suitable couplings. The housing electrical couplings 350 are configured to be coupled to the electrical portion 208, which can include the housing electrical connectors 336 and mating electrical port connectors (not shown) to communicatively couple the controller 350 (or 250) to the first sensing component 210, the second sensing component 220 and/or any other sensors or actuators positioned on the garment 200 or any other garment described herein.

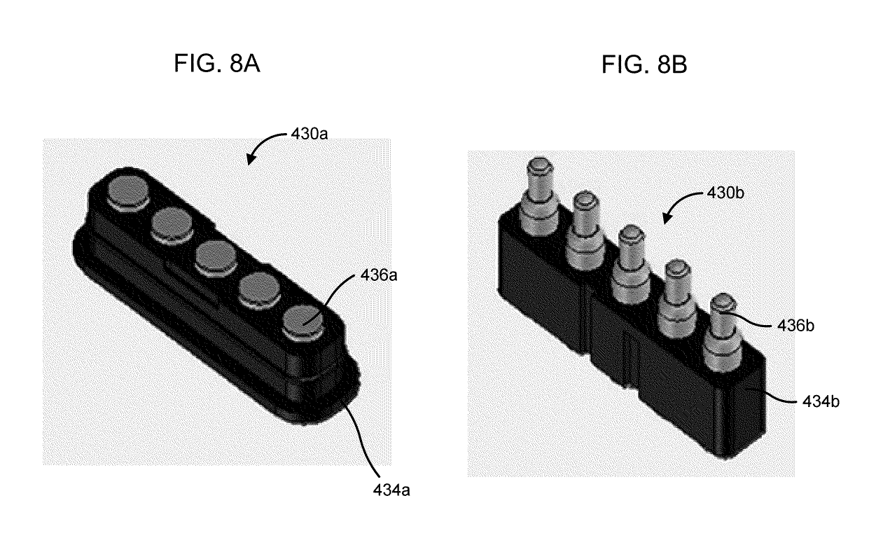

As described above, in various embodiments, the garment 200 also includes an attachment mechanism to secure the device including the controller to the garment 200. In this manner, the attachment mechanism establishes a connection between the electrical port of the garment 200 which can include a plurality of electrical port connectors. For example, FIG. 8A shows an embodiment of a housing electrical connector 340a which can be used to electrically couple the controller 250 or 350 to the first sensing component 210, the second sensing component 220 and/or any other sensors or actuators positioned on the garment 200 or any other garment described herein. The housing electrical coupling 340a includes a base 434a and a plurality of flat electrical connectors 436a configured to be coupled to corresponding connectors included in the electrical port 208 of the garment 200. In various embodiments, the electrical port 208 of the garment 200 can include electrical connectors which are substantially similar to the housing electrical connector 340a. One or more housing electrical connectors 340a can be provided on the housing 330 or otherwise the controller 350 or 350.

In various embodiments, the one or more housing electrical connectors 340a can be magnetic so that the housing electrical couplings 340a can magnetically attach to the electrical port connectors and thereby, be electrically coupled to the electrical port 208 of the garment 200. FIG. 8B shows another embodiment of a housing electrical coupling 340b which includes a base 346b with a plurality of pin connectors 344b positioned thereon. In various embodiment, the plurality of pin connectors 344b are configured to be inserted into female sockets provide in the electrical port 208. In other embodiments, the pin connectors 344b include spring loaded pogo pin connectors. In such embodiment, the electrical port can 208 include flat contact pad type connectors or terminals which are contacted by the pogo pin connectors 344b for communicatively coupling the electrical port 208 and thereby, the first sensing component 210, the second sensing component 220 and/or any other sensors or actuators positioned on the garment 200 or any other garment 200 described herein to the controller 250 or 350.

In some implementations, the housing 330 of the controller 350 may include a magnet that is configured to magnetically attach to a magnetizable portion of a housing of the electrical port 208. In some implementations, the housing of the electrical port 208 may include a magnet configured to magnetically attach to the housing 330 of the controller 350. In this way, the device that includes the controller can be securely attached to the garment and easily removed by the wearer.

Referring again to FIG. 5, in some embodiments, one or more haptic vibrators 282 are also positioned on the garment 200 which are configured to receive a signal from the controller 250 responsive to the controller 250 or the garment management system 120 detecting a trigger event. The trigger event can be based on the resistance value of the first sensing component 210 and/or the second sensing component 220. For example, the controller 250 determines a poor posture or improper breathing pattern of a wearer of the garment 200 from the resistance signal provided by the first sensing component 210 and the second sensing component 220. In response to the determination, the controller 250 can send an actuating signal to the one or more haptic vibrators 282 to alert the wearer of the decline in performance, under exertion or over exertion. In various embodiments, the one or more haptic vibrators 282 are positioned at a second location of the garment 200 corresponding to a bony location on the torso of the wearer, for example a collarbone of the wearer when the wearer wears the garment 200, and/or a wrist of the wearer. In some implementations, the haptic vibrators can be positioned at hip bones, knees, ankles, elbows, shoulder blades, among other bones where the vibrations can be felt. In some implementations, the intensity of the vibrations can be controlled such that they are noticeable by the wearer but high enough to invoke pain through the trigger of sensory pain nerves. In some implementations, the triggering events and the sensors 282 are similar to the events and sensors described above with respect to FIGS. 1-3.

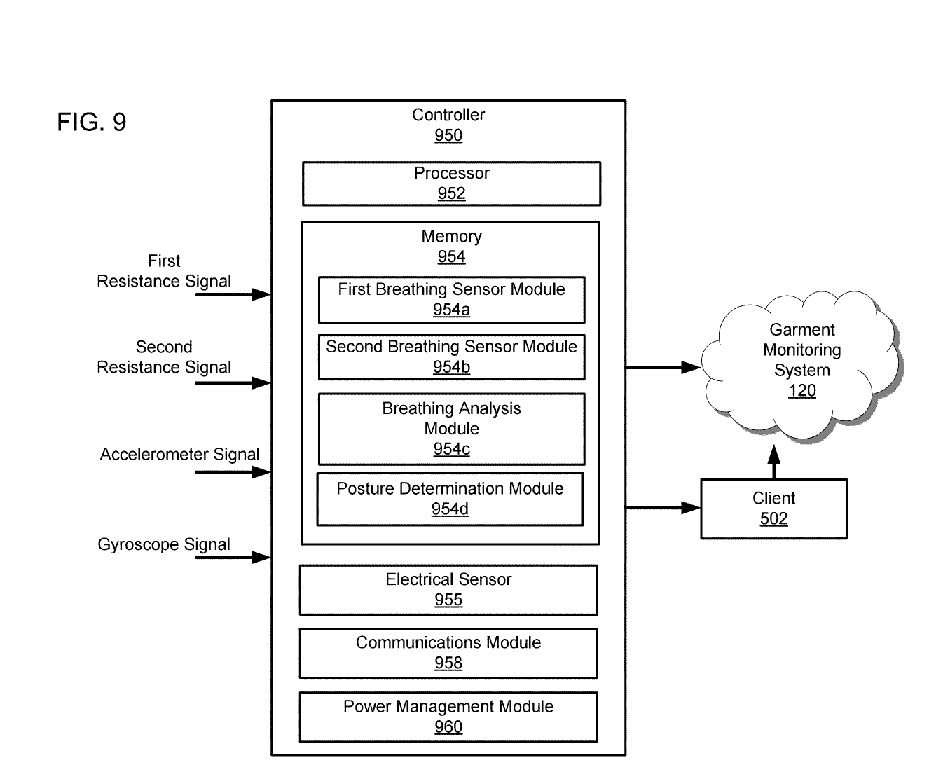

FIG. 9 is a schematic block diagram of an environment that includes a controller, a client device, and one or more servers capable of communicating with the controller via the cloud. As shown in FIG. 9, a controller 950, similar to the controller 950, can include a processor 952, a memory 954 or other computer readable medium or any other memory described with respect to the computer device 100 herein. The memory can store computer-executable instructions. The memory 954 can include a first breathing sensor module 954a, a second breathing sensor module 954b, a breathing analysis module 954c and a posture determination module 954d, among other modules. The controller 950 can also include one or more sensors 955, a communications module 958 and a power management module 960.

The processor 952 can include a microprocessor, programmable logic controller (PLC) chip, an ASIC chip, or any other suitable processor. The processor 952 is in communication with the memory 954 and configured to execute instructions, stored in the memory 974. In various embodiments, the processor 952 can be substantially similar to the CPU 121 or main processor 121 described herein with respect to FIGS. 4C-D, respectively.

The memory 954 includes any of the memory and/or storage components discussed herein. For example, memory 954 may include RAM and/or cache of processor 952. Memory 954 may also include one or more storage devices (e.g., hard drives, flash drives, computer readable media, etc.) either local or remote to device controller 950. The memory 954 is configured to store look up tables, algorithms or instructions. In various embodiments, the memory 954 can be substantially similar to the main memory 122 described with respect to FIGS. 4C-D herein.

The first breathing sensor module 954a is configured to provide functionality to allow the controller to receive a first resistance signal from the first sensing component of the garment. The first breathing sensor module 954a can be configured to cause the controller, via the power management module 960, to apply a voltage across the first sensing component. The power management module 960 can be hardware, software, or a combination of both hardware and software components, that is capable of applying a voltage across the first sensing component 210. The power management module 960 can be configured to apply a continuous voltage across the first sensing component 210. The voltage can be a fixed voltage, ranging from a few microvolts, to a few millivolts, to a few volts, or higher. In some implementations, the voltage can vary based on a power management policy configured to cause the first sensing component to provide resistance values that can be processed by the first breathing sensor module 954a.

The first breathing sensor module 954a can cause the controller to apply a voltage to the first sensing component 210. The first breathing sensor module 954a can cause the controller to apply the voltage via one or more electrical terminals coupled to one or more wires of the first sensing component. In some implementations, the first breathing sensor module 954a can cause the controller to receive a first resistance signal that includes resistance values across the first sensing component 210. The first breathing sensor module 954a can sample the first resistance signal at a predetermined frequency and store the resistance values of the first resistance signal. In some implementations, the first breathing sensor module 954a can sample the resistance values every 5 ms, 10 ms, 50 ms, 1 second, among other values.

In some implementations, the one or more sensors 955 can be configured to sense or otherwise determine resistance values from the first resistance signal received from the first sensing component. In some implementations, the sensors 955 can determine a first resistance value based on a voltage or current of the first resistance signal. In some implementations, the one or more sensors 955 can be configured to sense or otherwise determine resistance values from the second resistance signal received from the second sensing component. In some implementations, the sensors 955 can determine a second resistance value based on a voltage or current of the second resistance signal. The values determined by the sensors can be provided or accessed by the first breathing sensor module 954a and the second breathing sensor module 954b to determine breathing related data as described herein.

In some implementations, the first resistance signal can include voltage or current values. The first breathing sensor module 954a may include instructions to determine a resistance value based on the voltage or current values included in the first resistance signal. In some implementations, the resistance values determined from the first resistance signal can be used to determine a length of the first sensing component 210. The resistance value can be greater when the first sensing component is stretched, which would occur when the wearer's chest is expanded due to air in the lungs, indicative of the wearer inhaling. Conversely, the resistance value can be lower when the first sensing component is relaxed, which would occur when the wearer's chest is relaxed, indicating that the air in the chest has been exhaled).

The memory 974 includes a first sensing component module 954a which stores instructions configured to determine a first resistance or resistance change of the first sensing component 910 from the first resistance signal. For example, the sensor 956 can interpret the first resistance signal, for example a current or a voltage, and the first sensing component module 954a can use algorithms, equations (e.g., Ohm's law), reference or lookup tables, and/or current-voltage maps to determine the first resistance of the first sensing component 910. The memory 954 also includes a second sensing component module 954b which stores instructions configured to determine a second resistance or resistance change of the second sensing component 920 from the second resistance signal. For example, the sensor 956 can also interpret the second resistance signal, for example a current or a voltage, and the second sensing component module 954b can use algorithms, equations (e.g., Ohm's law), reference or lookup tables or current-voltage maps to determine the second resistance of the second sensing component 920.

The first breathing sensor module 954a can include a calibration routine to calibrate the first sensing component 210. Additional details regarding the calibration process are provided below. However, via the calibration process, the controller 950 and the first breathing sensor module 954a can identify a state when the first sensing component is at its maximum length, which would occur when the wearer has inhaled air. The first breathing sensor module 954a can record or otherwise identify a resistance value of the first sensing component at this maximum length. Similarly, the first breathing sensor module 954a can identify a state when the first sensing component is at its minimum length, which would occur when the wearer has exhaled the air. The first breathing sensor module 954a can record or otherwise identify a resistance value of the first sensing component at this minimum length. By identifying the two resistance values, the first breathing sensor module 954a can use these resistance values are guideposts or markers to identify what phase of a breathing routine a wearer is in based on the resistance values. If the resistance values are increasing over time, the first breathing sensor module 954a can determine that the wearer in inhaling. Conversely, if the resistance values are decreasing, the breathing sensor module 954a can determine that the wearer is exhaling. Further, depending on the resistance value relative to the maximum resistance value and the minimum resistance value, the first breathing sensor module 954a can determine where the wearer is during a breathing cycle.

The second breathing sensor module 954b is similar to the first breathing sensor module 954a but is configured to determine a resistance value based on the voltage or current values included in the second resistance signal received from the second sensing component 220. The second breathing sensor module 954b can go through a similar calibration process to identify a maximum and minimum length and corresponding resistance values at those lengths.

Various breathing related analytics can be performed based on the resistance values of the first sensing component 210 and the second sensing component 220. In some implementations, one or more additional sensors in communication with the controller can provide additional information for analyzing breathing. A sensor to detect electrical activity of the heart can further be used for analyzing breathing.

In some implementations, the breathing analysis module 954c can be used to analyze the breathing based on the signals received by the controller 950. In some implementations, the first breathing sensor module 954a and the second breathing sensor module 954b may be configured to collect the resistance values, while the breathing analysis module 954c can analyze the breathing based on the values determined by the first breathing sensor module 954a and the second breathing sensor module 954b of the controller 950.

In some implementations, the breathing analysis module 954c may include instructions to analyze breathing based on the resistance values received from the first sensing component 210 and the second sensing component 220. In some implementations, the breathing analysis module 954c can be configured to determine a breathing pattern of the wearer based on the rate of change of resistance values received from one or more of the first sensing component and the second sensing component. Further, the breathing analysis module 954c can be configured to determine a breathing quality metric based on the resistance values received from the first sensing component and the second sensing component. The relationship of the expansion and contraction of the chest and the abdomen can be used to determine a breathing quality of the wearer. As such, the breathing analysis module 954c can monitor the resistance values corresponding to the first sensing component (chest) and the second sensing component (abdomen) to determine the breathing quality. In some implementations, the breathing analysis module 954c can be executing on a server remote from the controller 950. In some implementations, the breathing analysis module 954c can be executing on a client device of the wearer. In some implementations, the breathing analysis module 954c can be executing on a server in the cloud and can receive the resistance values collected by the first and second breathing sensor modules 954a and 954b via the client device 502 or the controller 950 itself.

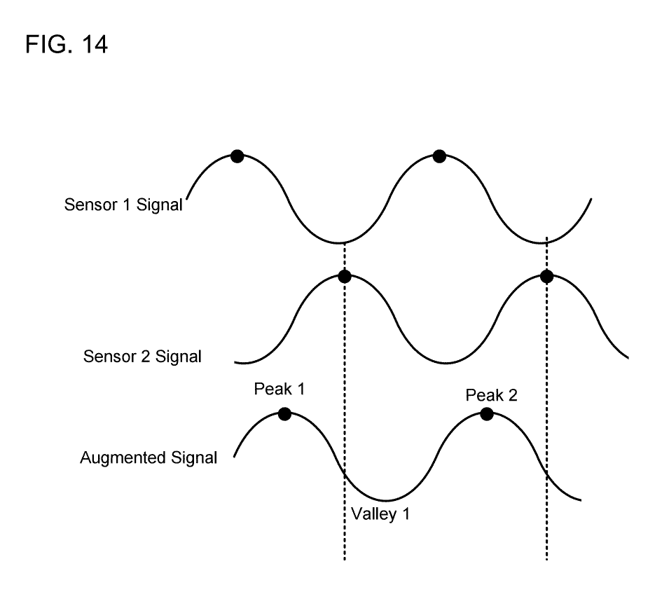

The breathing analysis module 954c may be configured to determine a breathing pattern of the wearer from the first resistance or resistance change of the first sensing component 910, and the second resistance or resistance change of the second sensing component 920. For example, the breathing analysis module 954c can include instructions, algorithms and/or lookup tables to determine an average or augmented resistance from the first resistance and the second resistance, analyze the change in resistance over time of the first sensing component 910 and the second sensing component 920 to determine a breathing phase, length of inhale and exhale, length chest inhale and exhale, length of abdomen inhale and exhale and/or determine a breathing quality of the wearer (e.g., for a wearer performing yoga), etc. In various embodiments, the breathing analysis module 954c can also map or chart a wearer's breathing patterns or quality over time. Additional details relating to the breathing analysis module 954c are provided below with respect to FIGS. 14-17

The controller 950 can also be configured to receive signals from the first sensor 260 (shown in FIG. 5A) which can include an accelerometer signal, and the second sensor 270 (shown in FIG. 5A) which can include a gyroscope, to determine a posture or orientation and/or motion of the wearer.

In various embodiments, the posture determination module 954d can be configured to receive and interpret signals from at least one accelerometer (e.g., the first sensor 960) and at least one gyroscope (e.g., the second sensor 970) to determine a posture or orientation of the wearer. For example, the posture determination module 954d can use the accelerometer and gyroscope signals to determine a position in space of the wearer, speed and/or orientation of the wearer's movements, stability of the wearer in maintaining a specific pose or position, or any other information related to the posture or orientation of the wearer. As described before with respect to FIGS. 5 and 6, the controller 950 can be positioned proximate to a spinal cord of the wearer when the garment 900 is worn by the wearer, for example, near a base of the neck of the wearer at the top edge 902 of the garment 900. This location is negligibly impacted by the expansion and contraction of the garment 900 at the first location (i.e., the pectoral region) and the second location (i.e., the abdominal region) and thus the signal from the accelerometer (i.e., the first sensor 960) and the gyroscope (i.e., the second sensor 970) can be relatively free of noise and/or false signals.

The controller 950 also includes a communications module 958 configured to communicate data to one or more devices via one or more wired or wireless connection such as Bluetooth®, Wi-Fi, RFID, NFC, or any other communication methodology described herein. For example, the communication module 958 can be configured to provide data for display to a remote computing device, such as a client device (for example, a mobile smartphone or tablet) that is capable of displaying data based on the sensing data received from the garment by the controller. In some implementations, the controller may transmit the data received from the sensor components of the garment. In some implementations, the controller 950 can process the data received from the sensor components and transmit data based on an analysis of the data received from the sensor components. The data based on the anaylsys, for example, a breathing pattern, chest breathing rate, abdominal breathing rate, overall breathing rate, breathing quality, posture quality, time, duration of physical activity, alerts, alarms (e.g., corresponding to an improper exercise routine or pose), long term breathing pattern, rewards, or any other information can be provided to the client device or a server executing on the cloud. In some embodiments, the communications module 958 is communicatively coupled to the one or more haptic vibrators 582. The communications module 958 can be configured to activate the haptic vibrators 582 and provide feedback to the wearer on the wearer's performance while performing the physical activity, or guide the wearer in improving his routine, as described before in detail herein.

In other embodiments, the communications module 958 can communicate breathing information and the posture and/or orientation information determined by the controller 950 to a client 502 or the cloud 508 (e.g., a remote server), for post processing and/or providing feedback information to the wearer. For example, the client 502 can include a smart phone, a tablet, a smart watch, a computer, a dedication fitness monitoring device or any of the clients 102a-n as described before with respect to FIG. 4A.

The client 502 can include an app or software for receiving the data from the controller 950 and analyzing the received data. The app can be configured to present, for display or some other sensory output, to the wearer data relating to the wearer's performance. In some implementations, the output can be information relating to the wearer's breathing patterns, movements, orientations, poses, muscle usage, among others. In some implementations, the data can include additional information, for instance, statistics relating to the wearer's sensed data. The data can be updated in near real-time as the data is being collected by the controller and transmitted to the client 502. The app or software can also be configured to store the wearer's performance over a period of time which can be communicated to the wearer in the form of a chart or a curve. The app or software stored on the client 502 can also provide feedback to the wearer, for example recommendations on improving the wearer's performance, nutritional information, exercise routines, or any other useful information. In various embodiments, the communications module 958 can also communicate data from other sensors, for example an ECG sensor, a breath sensor, a GPS, a light sensor, a salinity sensor or any other sensor which can be included in the garment 200 to the client 502 or the cloud 508. The data from the various sensors can be used to determine various physiometric parameters of the wearer as described in detail with respect to FIGS. 1-3.

The client 502 and/or server on the cloud 508 can include a memory storing computer-executable instructions to determine an overall health, fitness level and provide real-time feedback on the performance of the wearer while performing a physical activity, for example breathing patterns, breathing capacity, calories burned, water loss, posture, stability, location, etc. and/or develop trends to chart the wearers performance over an extended period of time. Additional details regarding the functionality of the client or the GMS 120 executing on the cloud are provided with respect to FIG. 10B.

FIG. 10A is a schematic block diagram of a cloud computing environment according to systems and methods described herein which includes a server executing on the cloud 508. The server can function as the garment monitoring system 120 and is communicatively coupled to a plurality of controllers 250a-250n corresponding to a plurality of garments 200 worn by a plurality of wearers, for example via a wired or wireless connection (e.g., Bluetooth®, Wi-Fi, RFID, NFC, or any other communication methodology described herein). In other embodiments, the garment monitoring system 120 can be communicatively coupled to a plurality of clients 502a-n configured to communicate information from the plurality of controllers 250a-n to the garment monitoring system 120 and/or receive information from the garment monitoring system 120 (e.g., software updates, feedback on wearer's performance, etc.).

Referring now to FIG. 10B, FIG. 10B shows a block diagram of the garment monitoring system (GMS) 120. The GMS 120 includes a wearer profile manager 1010, a sensor data manager 1020, a trend analyzer 1030, a wearer profile classifier 1040, a routine generator 1050 and a progress analyzer 1060. The GMS 120 also includes one or more databases, such as a wearer profile database 1012 and a routine database 1052.

The wearer profile manager 1010 can include hardware, software or a combination of hardware and software. In some implementations, the wearer profile manager 1010 can include computer-executable instructions to manage one or more profiles of wearers. The GMS can maintain a list of wearers in the wearer profile 1012. The wearer profile manager 1010 can be configured to create and update entries in the wearer profile database 1012. In some implementations, when a new wearer registers with the GMS 120, the wearer profile manager 1010 can create an entry in the database 1012 for that particular wearer. The wearer can register via a client device. In some implementations, the wearer can be registered with a particular controller. The controller can include a unique identifier through which activities performed by the wearer are tracked. The wearer profile manager can receive information relating to the wearer's weight, height, age, BMI, and other metrics. In some implementations, the wearer provides it to the wearer profile manager 1010. In some implementations, the GMS can determine the information using one or more sensors or devices communicatively coupled to the GMS. The wearer profile manager 1010 can communicate with one or more other modules of the GMS 120.

The sensor data manager 1020 can include hardware, software or a combination of hardware and software. In some implementations, the sensor data manager 1020 can include computer-executable instructions to manage sensor data passed to the GMS via one or more controllers. The sensor data manager 1020 can receive data packets from a controller. The sensor data manager 1020 can identify the controller corresponding to the data packets based on an identifier included in the data packets. In some implementations, the data packets can include information received from the sensing components and other sensors communicating with the controller. The sensor data manager 1020 can parse the data packets and identify resistance values, or other data that the controller transmits to the controller. The sensor data manager can manage this data and can update entries corresponding to a wearer to include the received data.

The sensor data received by the sensor data manager 120 can include raw data from the sensing components that are stored by the controller. In some implementations, the sensor data can be data that has been generated by the controller from the raw data of the sensing components. The sensor data can include breathing related data, posture or orientation related data, among others.

The trend analyzer 1030 can be configured to analyze trends based on the data received by the GMS 120 from one or more controllers. In some implementations, the trend analyzer can look at the breathing data received by or generated by the GMS 120 to identify particular trends. For instance, the trend analyzer can identify, from data received from a plurality of controllers, that a subset of the controllers had similar breathing pattern data and orientation data. From this data, the trend analyzer can determine that the wearers of the controller may have attended the same class or performed the same exercise.

The wearer profile classifier 1040 can be configured to classify wearers based on the data received from controllers corresponding to the wearers. In some implementations, the wearer profile classifier can analyze the data received from one or more controllers and determine that a particular wearer has low stamina. For instance, the wearer profile classifier can determine, from the data received, that the wearer's breathing indicates tiredness in an amount of time that is less than a predetermined threshold based on the routine the wearer performed (using position and orientation data). The wearer profile classifier can classify the wearers according to the types of exercises they perform, their skill level, their experience level, their strength level, their stability level, among others. In some implementations, the wearer profile classifier can be configured to utilize additional information from the wearer's profile database to identify wearer's similar to one another and within a predefined geographical area. In this way, the wearer profile classifier can identify a subset of wearers that may be suitable for a particular class or exercise offering within the predefined geographical area.

In some implementations, the routine generator 1050 can be configured to generate one or more exercise routines. The exercise routines can be based on orientation data received from a wearer. For instance, a wearer, such as yoga instructor, may perform a yoga routine. The controller can record values from the sensing components of the garment, including the position, motion and orientation sensors, and based on this data, generate an exercise routine. The exercise routine can be based on meeting certain parameters that can be sensed, for instance, certain orientations or postures for particular durations, breathing rates for particular durations, among others. The routine generator 1050 can store one or more routines 1052 in the routine database.

The progress analyzer 1060 can be configured to determine and track a progress of a wearer. The progress analyzer can identify previous data of the wearer and compare the previous data to newly received data from the wearer's controller. The data that is received from the controller can be broken down and analyzed to identify improvements in certain threshold metrics, for instance, core stability. This can be determined by identifying that the wearer is in a first orientation or posture, measuring an amount of movement (related to instability) in the wearer's ability to maintain the orientation or posture, and a length of time that the wearer can maintain the posture without exceeding a predetermined amount of movements related to instability. A wearer may increase the length of time the wearer can hold or maintain the pose without exceeding the instability motions over time, indicating progress. The progress analyzer can identify the progress based on comparisons of such metrics.

As described before with reference to FIGS. 5A and 6, the garment 200 can be made in any suitable size and configured to be worn by males or females. It is to be noted that the difference in overall torso length between the females and males is only about 10 cm. Therefore, a particular size of the garment 200 can be reliably used both by males and females. Furthermore, this can be used to create a standard fitting scheme to ensure accurate breathing data despite the variability of body sizes. Size “medium” can be used as a baseline for the first sensing component 210 and the second sensing component 220 placement and all other sizes are scaled according to the optimal distance from the neckline 202 to each sensor location on size medium. The scaling is +/−2 cm for distance d1 and +/−1.5 cm for distance d2 and is summarized in Table I:

TABLE I
Changes in positioning of first sensing component and
second sensing component on a garment based on size

| Size | d1 (cms) | d2 (cms) |
|---|---|---|
| Small (S) | −2 | −1.5 |
| Medium (M) | 0 (baseline) | 0 (baseline) |
| Large (L) | +2 | +1.5 |
| Extra Large (XL) | +4 | +3 |

Size differences internal to a given size, for example variability in body sizes within the large size category, may be compensated for by a combination of measuring the initial resistance of the first sensing component 210 and the second sensing component 220 during manufacturing and a calibration routine performed by the wearer once the wearer has received the garment that generates a maximum and minimum value for the wearer, as described in further detail herein. Circumferential difference within sizes is made up for by the stretch in the fabric or material forming the base component which has can have elastic stretchability of up to 20% within each size category.

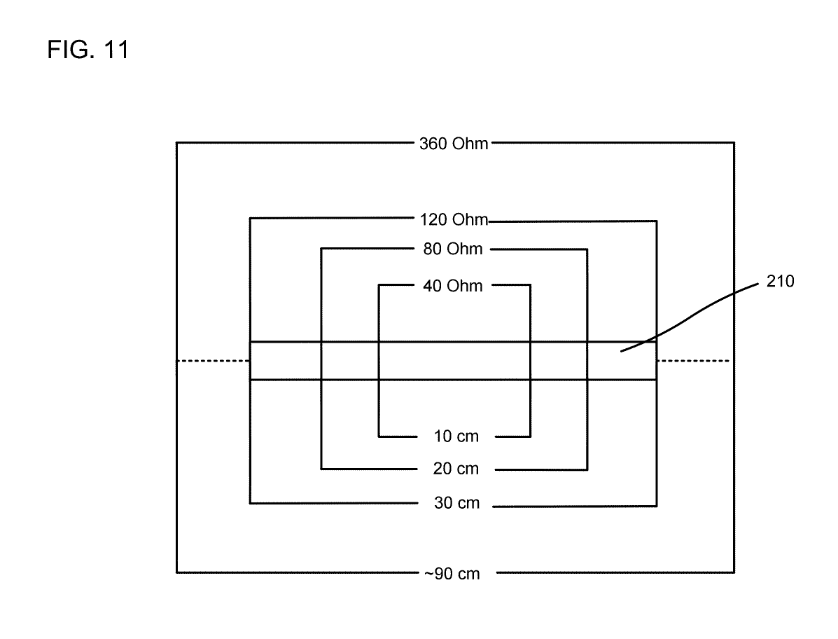

A change in length of the first sensing component 210 and the second sensing component 220 results in a change in resistance of the breathing sensors as shown in FIG. 11. The resistance of the breathing sensors is additive across the length. However, there may be variability in the resistance values in the underlying conductive fabric, i.e., the base layer 212 with the electrically conductive material 214 deposited thereon. There is also variability in the precise location of the first sensing component 210 and the second sensing component 220 located on the wearer, as well as variability in the circumference of individuals wearing the same size garment 200.

The variability in the baseline resistance values of the first sensing component 210 (e.g., the first sensing component 210) and the second sensing component 220 (e.g., the second sensing component 220) may be introduced during the manufacturing of the sensors, for example during a plating or coating of the base layer 212 with the electrical conductive material 214 (e.g., during a silver plating process on a stretchable base layer 212). A large roll of conductive fabric will have variability along both weft and warp of the fabric due to uneven coating or plating. This can make it difficult to ensure a reliable and repeatable sensor value across multiple sensing components formed from a single or multiple swaths of fabric. To mitigate this variability various steps can be performed including, for example: (1) the fabric strip forming the sensing components 210 and 220 can be precut to the desired width of the sensing components 210 and 220 before coating or plating with the electrically conductive material. This allows the coating or plating process to be applied to a smaller and more focused surface area, leaving less room for variability during the coating process; (2) the sensing components 210 and 220 being cut out of a larger swath of the first layer 212 fabric can have its resistance measured between the two ends of the sensor at 0%, 5%, 10%, 15%, 20% stretch. The values can then be stored as a reference table for each respective sensor in a respective garment; and (3) measure the resistance between multiple two points along the length of the sensing components 210 and 220.

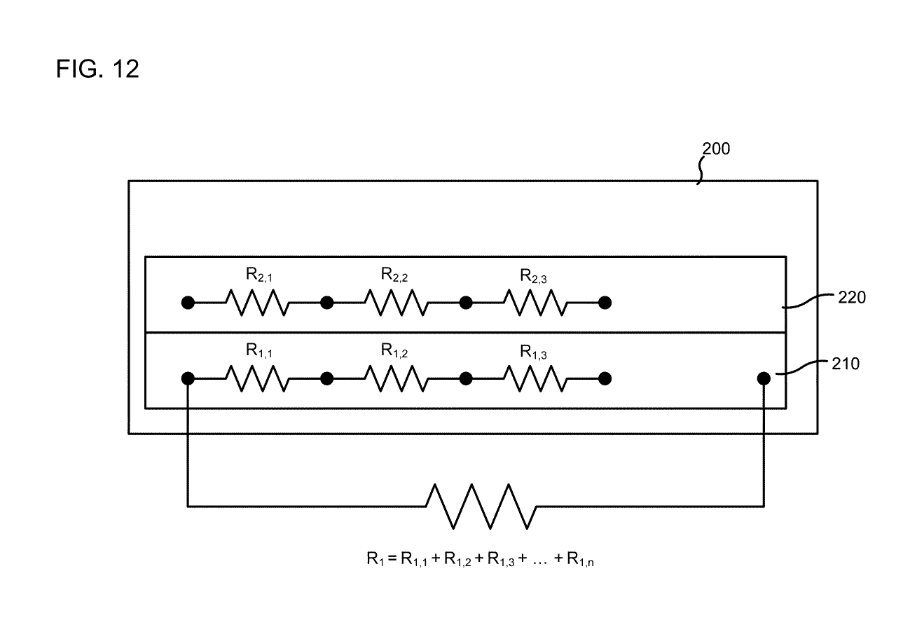

Expanding further on the third option, each of the sensing components 210 and 220 can be treated as multiple resistors arranged in series, the additive resistive of which corresponds to the overall resistance of the sensing components 210 and 220. For example, FIG. 12 schematically shows resistance values measured between a plurality of points on the first sensing component 210 and the second sensing component 220. The points are arranged at a predetermined distance from each other, for example equally spaced from each other. The resistance is measured between each of the adjacent points and the sum of all the resistances corresponds to the overall resistance. This provides the benefit of more discrete and thus accurate estimates of resistance change along the sensing component. Sampling from multiple points along the sensing component allows for normalization of the resistance variability along the length of the sensing component. This also provides a more robust way to filter out non-breath related stretch of the garment due to motion of the underlying body, by providing additional data points of stretch along the length of the sensing component. Table II summarizes measured resistances at 10 cm intervals along a stretch direction (warp) of a material used to form the first sensing component 210, as shown in FIG. 12.

TABLE II
Measured resistance at various points on a breathing sensor material
measured at between various points spaced apart by 10 cms

| R_{1,1} | R_{1,2} | R_{1,3} | R_{1,4} | R_{1,5} | R_{1,6} | R_{1,7} | R_{1,8} | R_{1,9} | R_{1,10} | R_{1,11} | R_{1,12} | R_{1,13} | R_{1,14} |
|---|---|---|---|---|---|---|---|---|---|---|---|---|---|
| Ω | Ω | Ω | Ω | Ω | Ω | Ω | Ω | Ω | Ω | Ω | Ω | Ω | Ω |
| 4.1 | 5.4 | 9.4 | 22.5 | 23 | 13.7 | 12.5 | 14.5 | 13 | 9.5 | 11.2 | 13.1 | 14.2 | 9 |

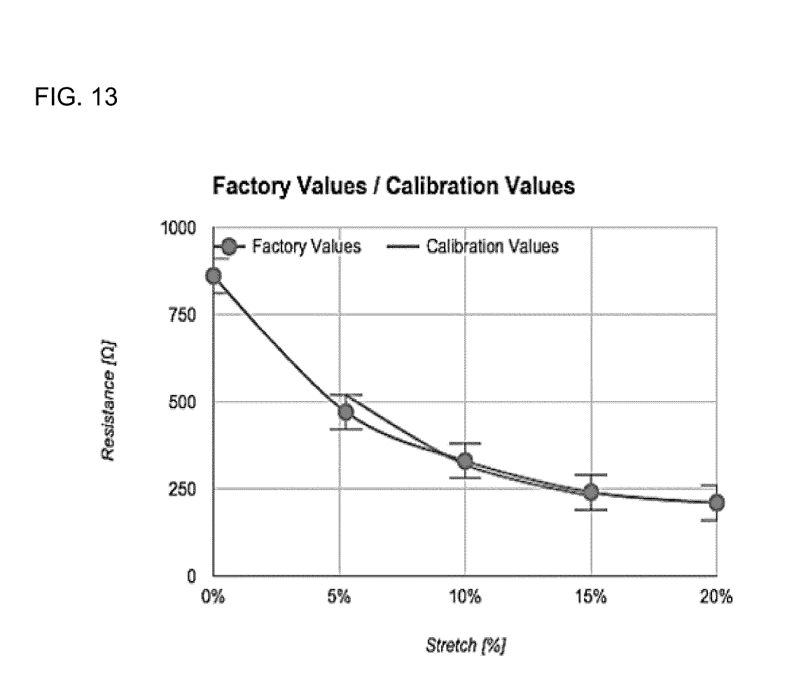

Referring to FIG. 13, the variability due to imprecise location of the breathing sensor on the body, as well as the variability of the circumference between wearers within a given size category can be mitigated by performing a calibration routine once the wearer has put the garment on. The calibration routine provides a maximum and minimum value for the breathing capacity of both the chest and abdominal cavity of the wearer. This allows comparison with known baseline resistance values recorded during manufacturing (curve labeled as “factory values” in FIG. 13) and thereafter, comparison with a database of known expected value ranges for a wearer wearing a given size garment. If the wearer self-reports their height and weight, this information can be used to make the calibration even more accurate.

In various embodiments, the calibration routine for the first sensing component 210 and/or the second sensing component 220 includes wearing the garment 200 on a torso of the wearer. The controller 250 of the garment 200 is connected to a client, for example a smartphone, a smartwatch or a tablet. The wearer stands upright, and inhales by taking a deep breath to cause expansion of the breathing sensors. Next the wearer exhales removing air from the lungs causing contraction of the breathing sensors and the inhaling is repeated, for example 2, 3, 4 or even more times. The measured resistance corresponding to expanded and contracted sensing component due to the inhaling and exhaling, respectively is averaged for all the repeats and a best fit is applied to the known factory values. The wearer than sits in a chair with straight back repeats the inhaling and exhaling for a predetermined number of times, for example 2, 3 4 or even more times. The resistance values obtained with the wearer sitting down are also averaged and a best fit is applied to the known factory values.

FIG. 13 shows resistance plots of resistance of a first sensing component positioned around the chest of a wearer of an example garment to demonstrate the close correspondence between factory values and calibration values of the resistance. A breathing sensor placed around a torso of wearer can have a functional range of 5% stretch at the smallest circumference when the wearer is at full exhale and up to 15% stretch at the max inhale. The results of the characterization show the results of the sensing component within this range and their relation to the known factory values. In comparison, a person in the same size category but with slightly more girth, can, for example be from 7% stretch at full exhale to 18% stretch at full inhale. Thus performing the calibration using the method described herein can address the variability in sizes of a wearer within the same size range.

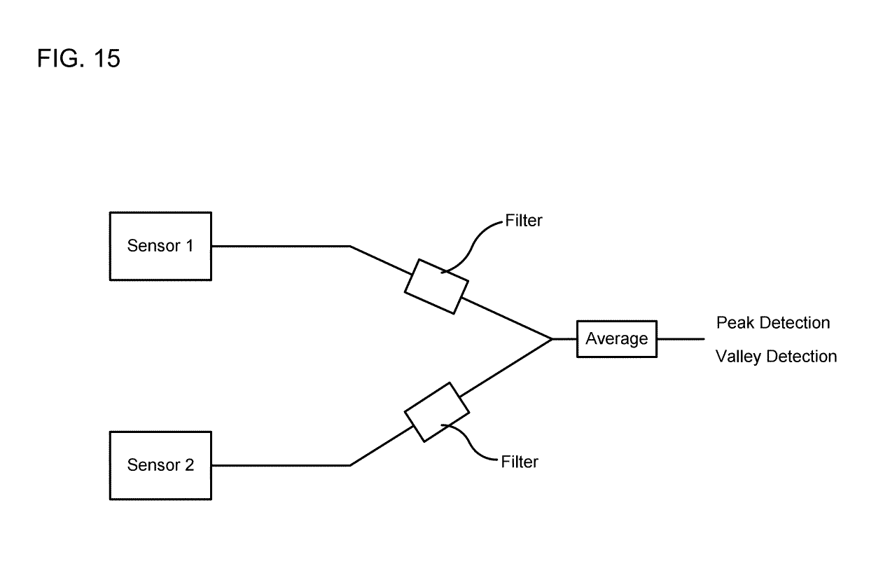

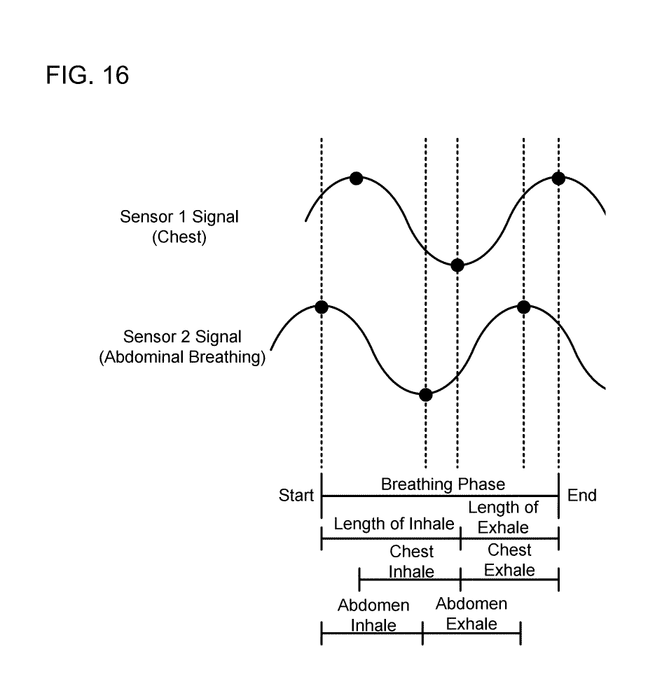

FIGS. 14 and 16 show real time resistance data obtained from a first sensor (also referred to herein as “sensor 1”) which can include the first sensing component 210, and a second sensor (also referred to herein as “sensor 2”) which can include the second sensing component 220. The breathing analyzer module 954c (shown in FIG. 9) can be configured to derive breathing rate and breathing pattern information from the resistance values generated or received from the first sensing component and the second sensing component 220. Resistance is read from sensor 1 positioned around the chest, and sensor 2 positioned around the abdominal region of the wearer, simultaneously. As shown in FIG. 15, the breathing analyzer module 954c can filter the resistance signals from sensor 1 and sensor 2 using any suitable filter (e.g., a low pass filter, a high pass filter, a band pass filter, or any other suitable filter). The breathing analyzer module 954c can then smooth the resistance values and normalize them. The breathing analyzer module 954c can then take an average of the resistance values from the two sensing components 210 and 220, for example using equation I:

$$\frac{SensorValue 1 ( n ) + SensorValue 2 ( n )}{2} = Average ( n ) \qquad ( I )$$

The peaks and valleys of the smoothed and normalized data from sensor 1 and sensor 2 are taken as well as of the averaged data (FIG. 14). The peak and valley for the averaged data is used to anchor peak 1 as the beginning of the inhale, valley 1 as the transition between inhale and exhale, and peak 2 as the end of the exhale. The breathing analyzer module 954c can use the corresponding peaks and valleys of sensor 1 and sensor 2 to assess the total time each breathing phase takes. The breathing analyzer module 954c can determine which of the two breathing sensors recorded a peak before the first average peak and identify that peak as the start of the breathing phase. The breathing analyzer module 954c can then determine whichever of the two sensors recorded a peak after the latter average peak and identify that peak as the end of the breathing phase. The breathing analyzer module 954c can determine the total time for each breathing phase and from this information based on the time between the two identified peaks. The breathing analyzer module 954c can determine or otherwise calculate other values such as length of inhale, length of exhale, time and relative volume of chest inhale and exhale, time and relative volume of abdomen inhale and exhale based on these values, as shown in FIG. 16. The breathing analyzer module 954c can be configured to detect a first peak before the first average peak and the second peak after the second average peak and based on detecting the first peak and the second peak, determine a total time for each breathing phase.

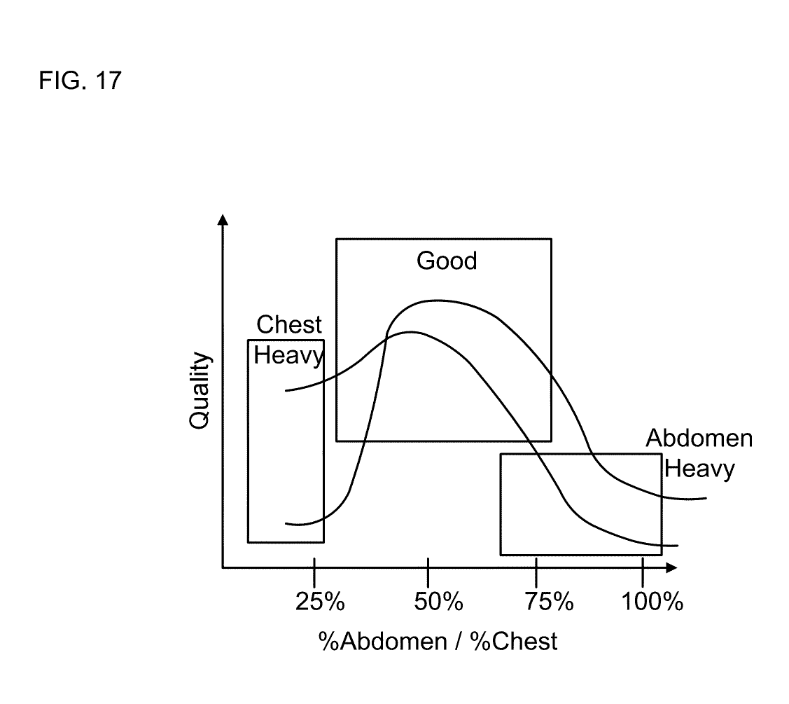

The breathing quality is then calculated by finding the ratio of the time of chest breathing to abdominal breathing within each breathing phase and is simply taken by finding the time between peaks of the chest signal and abdomen signal within the same breath phase and finding the ratio between them. For example, FIG. 17 shows sample resistance curves corresponding to sensor 1 (chest) and sensor 2 (abdomen). Different portions of the resistance curves correspond to breathing quality such as is the wearer breathing heavily from the chest, the abdomen or is maintaining a good breathing pattern which might include some predetermined amount of breathing from each of the chest and the abdomen.

Although in typical breathing patterns, the circumference of the torso increases as a user inhales and the circumference of the torso decreases as the user exhales, some users may have medical conditions in which this breathing pattern does not hold true. Instead, in some medical conditions, for instance, an abnormal orientation of the diaphragm, the circumference of the torso decreases as the user inhales and the circumference of the torso increases as the user inhales. The present disclosure and the garment described herein describe solutions to identifying such medical conditions. During the calibration routine, a wearer is asked to inhale and a change in resistance in the sensing components is recorded. The wearer is then asked to exhale and a change in resistance in the sensing components is recorded. The GMS 120 can compare the resistance values during the inhaling and exhaling phases and determine that the wearer′ chest is expanding during the exhale phase, while getting smaller during the inhale phase.

As described before the garment 200 is also configured to detect a posture or orientation or motion of the wearer using the first sensor 260 which can include an accelerometer and a second sensor 270 which can include a gyroscope. In various embodiments, the first sensor 260 can include a 3-axis digital accelerometer (e.g., ADXL362 from Analog Devices, Inc.) and a digital 3-axis gyroscope (e.g., L3GD20 from STMicroelectronics) for keeping track of the position of the wearer. The sampling rate can take place within a fixed period, for example of 50 millisecond (20 Hz) but any other sampling frequency can also be used. An inactivity threshold of the accelerometer can be set between a minimum and maximum G-force range, for example from 150 g (minimum) to 250 g (maximum). This ensures from the minimum threshold that the component stops making measurements if the wearer is in a still position, thereby saving energy. If the motion sensing is above 250 g, this is attributed as a noise or a malfunction, and measurements are stopped. A timing feature can also be included for these out of range measurements so that measurements are only stopped if the out of range measurements are recorded for at least a predetermined amount of time or a predetermined number of measurements. This allows the controller 250 to sense out of range for a period of time or samples occurrences to confirm that the measurements are actually due to malfunction and not an occasional glitch. In particular embodiments, the predetermined number of measurements can be 30. The accelerometer can be set in loop mode so that the accelerometer is always autonomously sampling within an Output Data Rate (ODR), for example of 100 Hz, without the intervention of the controller 250. The gyroscope can include an embedded passband and high-pass filters for pre-filtering the data. In particular embodiments, the gyroscope is operated at sampling rate of 20 Hz, a passband filter cutoff frequency of 12.5 Hz, to high-pass filter cutoff frequency of 0.9 Hz, and an Output Data Rate (ODR) of 95 Hz.

Data can be collected every 50 millisecond by request which can include, for example the 8 most significant bits from the accelerometer and the 16 most significant bits from the gyroscope. This information can be stored in a local buffer, for example, the memory 254 of the controller 250, along with the breathing sensors data and the battery level data on the controller 250. In various embodiments, when the local buffer is full, the data is communicated to a client device, for example a smart phone using wireless communication (e.g., low powered Bluetooth®).

The data from the accelerometer and the gyroscope is used to calculate the relative angles of the wearer with the ground as a reference. For this, all the axis' information from the accelerometer can be combined to find the vector that is being formed by the acceleration forces acting on the accelerometer. The gyroscope data which provides relative angular velocity for each axis, is incorporated into the accelerometer data. An indefinite integral can be applied to the data from the gyroscope to accurately measure small changes in the position and assess for any possible drift in the accelerometer data.

For the position recognition or otherwise posture detection of the wearer, the data from the accelerometer and the gyroscope can be compared with a reference library which can include various positions and movements and commonly associated angles therewith stored in a 2D array. Such positions can include, for example up right position, bend forward, bend backward, side to side, lying down, lying upside down, standing upside down, sitting, etc. Similarly, various movements can include standing up, sitting down, lying down, forward bending, backward bending, rising up, etc. A tolerance angle can also be used set the range and the accuracy of the recognition. The gyroscope data can then be used to keep track of the transition between each position, allowing determination of the exact position of the torso of the wearer at all times.

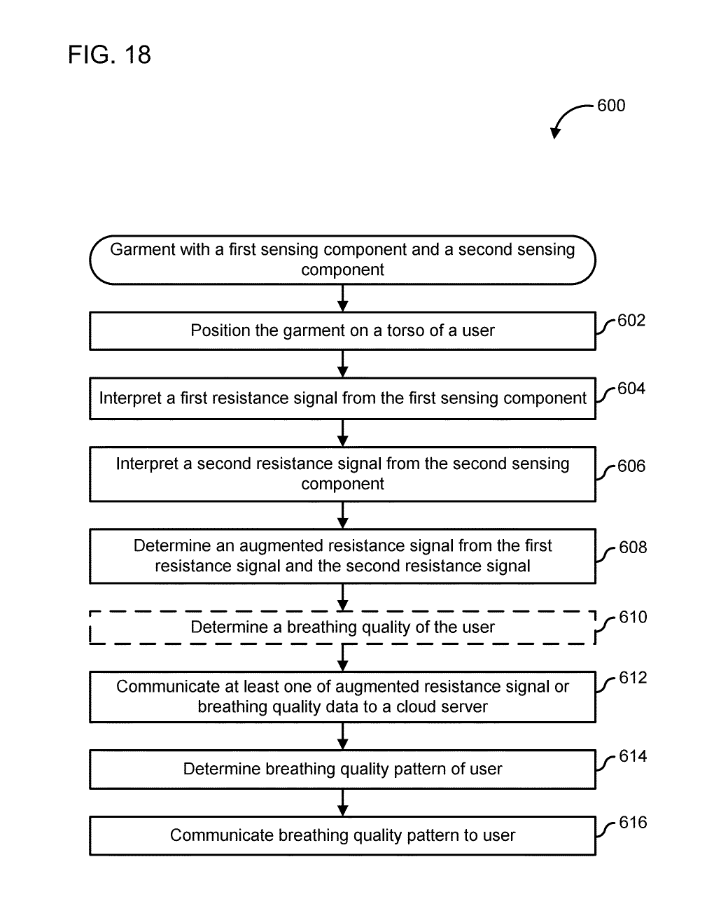

FIG. 18 is a schematic flow diagram of a method 600 for monitoring breathing pattern of a wearer using a garment wearable on a torso of the wearer. The garment includes a first sensing component configured to positioned proximal to a pectoral region of the wearer and a second sensing component configured to be positioned around an abdominal region of the wearer and can, for example include the garment 200 or any other garment described herein.

The method 600 includes positioning the garment on a torso of a wearer at 602. For example, the garment includes the garment 200 which a wearer wears on the torso of the wearer so that the first sensing component 210 of the garment 200 is positioned circumferentially around a chest of the wearer, and the second sensing component 220 of the garment 200 is positioned circumferentially around the abdomen of the wearer.

A first resistance signal from the first sensing component is interpreted at 604. A second resistance signal from the second sensing component is interpreted at 606. An augmented resistance signal is determined from the first resistance signal and the second resistance signal at 608. For example, the controller 250 can interpret the first resistance signal from the first sensing component 210 and the second resistance from the second sensing component 220 to determine a chest and abdomen breathing pattern of the wearer as described before herein. The controller 220 can then determine the augmented resistance signal which can include an average of the two signals to determine an overall breathing pattern of the wearer, as described before herein.

In some embodiments, a breathing quality of the wearer can be determined at 610. For example, the controller 250 can also include instructions (e.g., stored on a memory 254 of the controller) configured to determine a breathing quality of the wearer, for example if the wearer is breathing good, chest heavy, abdomen heavy or any other breathing quality described herein.

At least one of the augmented resistance signal or a breathing quality data is communicated to a cloud server at 612. For example, the controller 250 can either communicate the augmented or average resistance signal obtained from the first sensing component 210 and the second sensing component 220 to the cloud server which can, for example, include the cloud server 108 or 508, or communicate the breathing quality data determined from the augmented resistance signal to the cloud server. A breathing quality pattern of the wearer is determined at 614. For example, the cloud server 508 can include the breathing rate analyzer 516 for determining a breathing rate or breathing pattern of the wearer as described in detail with respect to FIG. 10.

In various embodiments, the cloud (e.g., the cloud 508) can also receive augmented resistance signals or breathing quality data from a plurality of controllers communicatively coupled to the cloud. In such embodiments, the cloud, for example the breathing rate analyzer module 516 included in the cloud 508 can compare the breathing quality data of the wearer against the breathing quality of one or more wearers corresponding to the plurality of controllers to provide information on how the breathing quality of the wearer compares to various other wearers and any other information, as described before herein. The breathing quality pattern is communicated to the wearer at 616. For example, the cloud 508 can communicate the breathing pattern of the wearer to the controller 250. The controller 250 includes a communications module 258 which can include audio or visual communication means (e.g., a display, speakers, etc.) for communicating the breathing pattern information to the wearer. In other embodiments, the GMS 120 can communicate the breathing pattern information to a client, for example, a smartphone, a smartwatch, a tablet, a computer or any of the clients 102a-n or 502a-n, as described before herein. The wearer can then access the client device to obtain the breathing pattern information or any other information pertaining to a physical activity of the wearer.

References to “or” may be construed as inclusive so that any terms described using “or” may indicate any of a single, more than one, and all of the described terms.

It should be noted that the term “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Additionally, it should be understood that features from one embodiment disclosed herein may be combined with features of other embodiments disclosed herein as one of ordinary skill in the art would understand. Other substitutions, modifications, changes and omissions may also be made in the design, operating conditions and arrangement of the various exemplary embodiments without departing from the scope of the present invention.

While this specification contains many specific embodiment details, these should not be construed as limitations on the scope of any inventions or of what may be claimed, but rather as descriptions of features specific to particular embodiments of particular inventions. Certain features described in this specification in the context of separate embodiments can also be implemented in combination in a single embodiment. Conversely, various features described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain embodiments, multitasking and parallel processing may be advantageous.

What is claimed is:

1. A garment for measuring one or more parameters of a wearer, comprising:
a base material configured to be worn by a wearer;
a sensing component having a first elastic stretchability along a first axis and a second elastic stretchability along a second axis that is greater than the first elastic stretchability, the sensing component integrated into a first location of the base material corresponding to a predetermined region of a wearer, the sensing component including an electrically conductive material having an electrical resistance that changes with a change in a length of the sensing component, the sensing component including at least one wire to electrically couple the electrically conductive material to a controller including a processor and a memory, the memory storing processor-executable instructions to cause the controller to determine a electrical resistance value across the sensing component via the at least one wire.

2. The garment of claim 1, wherein the base material includes a torso portion configured to surround a torso of a wearer, and wherein the first location of the base material is located within the torso portion of the garment.

3. The garment of claim 2, wherein the sensing component extends along a circumference of the torso portion and having a height below a predetermined threshold.

4. The garment of claim 1, wherein the base material is shaped and sized to form a shirt and wherein the first location of the base material into which the sensing component is integrated is positioned at a first distance from a neckline of the base material, the first distance based on a size of the base material.

5. The garment of claim 1, further comprising:
an electrical port coupled to the at least one wire, the electrical port positioned on a back portion of the garment that is configured to cover a back of the wearer; and
an attachment mechanism to secure a device including the controller and a connector to the garment and establish a connection between the electrical port and the connector.

6. The garment of claim 5, wherein the device includes a body orientation detection sensor and the attachment mechanism is positioned on the back portion of the garment that is aligned with a spinal column of the wearer when the garment is worn by the wearer.

7. The garment of claim 1, wherein the base material is shaped to be worn as a shirt, the sensing component includes a first sensing component integrated into the first location of the shirt that is a first distance from a neckline of the shirt, the first location corresponding to a pectoral region of the wearer when the wearer wears the shirt and wherein the shirt includes a second sensing component integrated into a second location of the shirt that is a second distance from the neckline of the shirt, the second location of the garment corresponding to an abdominal region of the wearer when the wearer wears the shirt.

8. The garment of claim 7, wherein the first sensing component is configured to measure a contraction and expansion of a rib cage and chest cavity of a wearer.

9. The garment of claim 7, wherein the second sensing component is configured to measure a contraction and expansion of an abdominal cavity of a wearer.

10. The garment of claim 1, wherein the sensing component includes a plurality of electrically conductive particles, the electrically conductive particles positioned between a first film and a second film, the second film having a water solubility below a predetermined threshold.

11. The garment of claim 1, wherein the sensing component includes a strip positioned in between a first film and a second film, and at least one of the first film or the second film is permanently secured to the garment.

12. The garment of claim 6, wherein the body orientation detection sensor includes at least one of an accelerometer, a magnetometer or a gyroscope, the controller configured to sample values from the posture detection sensor at a predetermined frequency, the posture detection sensor configured to communicate with the controller to determine a body orientation of the wearer.

13. The garment of claim 1, further comprising one or more haptic vibrators, the haptic vibrators configured to receive a signal from the controller responsive to the controller detecting a trigger event based on the resistance value of the sensing component.

14. The garment of claim 13, wherein the one or more haptic vibrators are positioned at a second location of the garment corresponding to a location of a bone of the wearer when the wearer wears the garment.

15. The garment of claim 14, wherein the one or more haptic vibrators are positioned at a second location of the garment corresponding to a location of a collarbone of the wearer when the wearer wears the garment.

16. The garment of claim 1, wherein the sensing component is positioned at a location of the garment to determine one of i) an expansion or contraction of a muscle or ii) a change in an orientation of a joint.

17. A shirt for measuring one or more parameters of a wearer, comprising:
a base material configured to be worn by a wearer;
a sensing component having a first elastic stretchability along a first axis and a second elastic stretchability along a second axis that is greater than the first elastic stretchability, the sensing component integrated into a first location of the base material corresponding to a predetermined region of a wearer, the sensing component including an electrically conductive material having an electrical resistance that changes with a change in a length of the sensing component, the sensing component including at least one wire to electrically couple the electrically conductive material to a controller including a processor and a memory, the memory storing processor-executable instructions to cause the controller to determine a electrical resistance value across the sensing component via the at least one wire.

18. The shirt of claim 17, wherein the sensing component extends along a circumference of the torso portion and having a height below a predetermined threshold.

19. The shirt of claim 17, further comprising:
an electrical port coupled to the at least one wire, the electrical port positioned on a back portion of the garment that is configured to cover a back of the wearer; and
an attachment mechanism to secure a device including the controller and a connector to the garment and establish a connection between the electrical port and the connector.

20. The shirt of claim 17, wherein the device includes a body orientation detection sensor and the attachment mechanism is positioned on the back portion of the garment that is aligned with a spinal column of the wearer when the garment is worn by the wearer.
